(19) United States
(12) Patent Application Publication
(10) Pub. No.: US 2026/0308270 A1
(43) Pub. Date: Oct. 8, 2026

(54) METHODS OF ENHANCING ANTIBODY THERAPIES

(71) Applicant: Ohio State Innovation Foundation, Columbus, OH (US)

(72) Inventors: Susheela TRIDANDAPANI, PlainCity, OH (US); Jonathan BUTCHAR, Hilliard, OH (US); Giovanna MERCHAND REYES, Columbus, OH (US); Ramasamy SANTHANAM, Columbus, OH (US); Payal MEHTA, Zionsville, OH (US)

(21) Appl. No.: 19/482,634

(22) PCT Filed: May 8, 2024
(86) PCT No.: PCT/US2024/028358

§ 371 Date: Nov. 7, 2025

Related U.S. Application Data
(60) Provisional application No. 63/500,961, filed on May 9, 2023.

(51) Int. Cl. C07K 16/28; A61K 38/05; A61K 38/06; A61K 39/00; A61P 37/04

(52) U.S. Cl. CPC C07K 16/2887; A61K 38/05; A61K 38/06; A61P 37/04; A61K 2039/505

(57) ABSTRACT

The present disclosure relates to immunotherapeutic compositions and methods of use thereof to treat and/or prevent cancer using a nucleotide-binding oligomerization domain-containing protein 2 (NOD2) agonist and an immunotherapeutic agent.

### CROSS-REFERENCE TO RELATED APPLICATIONS

This application claims priority to, and the benefit of, U.S. Provisional Patent Application No. 63/500,961, filed May 9, 2023, entitled “Methods of Enhancing Antibody Therapies using a NOD2 Agonist,” which is incorporated by reference herein in its entirety.

### STATEMENT REGARDING FEDERALLY SPONSORED RESEARCH

This invention was made with Government Support under Grant No. CA203584 awarded by the National Institutes of Health. The Government has certain rights in the invention.

### REFERENCE TO SEQUENCE LISTING

The sequence listing submitted on May 8, 2024, as an .XML file entitled “103361-495WO1_ST26” created on Apr. 25, 2024, and having a file size of 13,621 bytes is hereby incorporated by reference pursuant to 37 C.F.R. § 1.52(e)(5).

### FIELD

The present disclosure relates to immunotherapeutic compositions and methods of use thereof to treat and/or prevent cancer using a nucleotide-binding oligomerization domain-containing protein 2 (NOD2) agonist and an immunotherapeutic agent.

### BACKGROUND

Therapeutic antibodies have become a major strategy to treat oncologic diseases. For chronic lymphocytic leukemia, antibodies against CD20 are used to target and elicit cytotoxic responses against malignant B cells. However, efficacy is often compromised due to a suppressive microenvironment that interferes with cellular immune responses. To overcome this suppression, agonists of pattern recognition receptors have been studied which promote direct cytotoxicity or elicit anti-tumoral immune responses. NOD2 is an intracellular pattern recognition receptor that participates in the detection of peptidoglycan, a key component of bacterial cell walls. This detection then mediates the activation of multiple signaling pathways in myeloid cells. Although several NOD2 agonists are being used worldwide, the benefit of these agents in the context of antibody therapy has not been explored. Herein, it is shown that treatment of peripheral blood mononuclear cells with L18-MDP results primarily in the activation of monocytes. This was observed in cells from both healthy donors and CLL patients. Upon NOD2 stimulation, there was an upregulation of activating FcγR in monocytes and a subsequent increase in antibody-mediated phagocytosis. This effect required the NF-κB and p38 signaling pathways. Using the Eμ-TCL engraftment model of CLL, combining NOD2 agonists with a murine anti-CD20 antibody led to a significant reduction of CLL load, as well as to a phenotypic change in splenic monocytes and macrophages. Taken together, these results show that NOD2 agonists help overturn the suppression of myeloid cells, and augments the efficacy of antibody therapy for CLL.

Given the limitations described above, there is a need to develop a therapeutic composition and methods of use thereof to treat and/or prevent cancer, including but not limited to CLL.

### SUMMARY

The present disclosure provides immunotherapeutic compositions and methods of use thereof for treating and/or preventing a cancer, including but not limited to chronic lymphocytic leukemia (CLL). The present disclosure also provides methods of stimulating a monocyte or macrophage using a nucleotide-binding oligomerization domain-containing protein 2 (NOD2) agonist and an immunotherapeutic agent, including but not limited to a monoclonal antibody (mAb).

In one aspect, disclosed herein is a method of treating a chronic lymphocytic leukemia (CLL), the method comprising administering to a subject in need thereof a therapeutically effective amount of a nucleotide-binding oligomerization domain-containing protein 2 (NOD2) agonist and an immunotherapeutic agent, wherein the method decreases tumor burden of CLL relative to an untreated control.

In one aspect, disclosed herein is a method of treating a chronic lymphocytic leukemia (CCL), the method comprising administering to a subject a therapeutically effective amount of a composition comprising a nucleotide-binding oligomerization domain-containing protein 2 (NOD2) agonist, an immunotherapeutic agent, and a pharmaceutically acceptable carrier, wherein the method decreases tumor burden of CLL.

In one aspect, disclosed herein is a method of stimulating a monocyte or a macrophage, the method comprising exposing the monocyte or the macrophage to an effective amount of a nucleotide-binding oligomerization domain-containing protein 2 (NOD2) agonist and an immunotherapeutic agent, wherein the method shifts the monocyte or the macrophage from an M2-like phenotype to an M1-like phenotype.

In some embodiments, the NOD2 agonist comprises a muramyldipeptide (MDP), a muramyltripeptide (MTP), L18-MDP, M-TriLYS, murabutide, N-Glycolyl-MDP, or a derivative thereof. In some embodiments, the immunotherapeutic agent comprises a monoclonal antibody. In some embodiments, the monoclonal antibody binds to a tumor cell and interacts with a constant fragment (Fc) on an effector cell. In some embodiments, the NOD2 agonist and the immunotherapeutic agent are administered concurrently. In some embodiments, the NOD2 agonist and the immunotherapeutic agent are administered consecutively.

In some embodiments, the NOD2 agonist and the immunotherapeutic agent increases an antibody-mediated response relative to the immunotherapeutic agent alone. In some embodiments, the antibody-mediated response comprises monocyte or macrophage activation. In some embodiments, the method shifts a monocyte or a macrophage from an M2-like phenotype to an M1-like phenotype.

In some embodiments, the method further comprises administering an adjuvant, an additive, or a combination thereof. In some embodiments, the adjuvant, additive, or a combination are administered concurrently or consecutively with the NOD2 agonist and the immunotherapeutic agent. In some embodiments, the pharmaceutically acceptable carrier comprises an excipient, a diluent, a filler, a salt, a buffer, a stabilizer, a solubilizer, a lipid, a stabilizer, or any combination thereof.

In one aspect, disclosed herein is an immunotherapeutic composition comprising a muramyldipeptide (MDP), a muramyltripeptide (MTP), L18-MDP, M-TriLYS, murabutide, N-Glycolyl-MDP, or a derivative thereof, a monoclonal antibody; and a pharmaceutically acceptable carrier of any preceding aspect.

In some embodiments, the immunotherapeutic composition further comprises an adjuvant, an additive, or a combination thereof.

### BRIEF DESCRIPTION OF FIGURES

The accompanying figures, which are incorporated in and constitute a part of this specification, illustrate several aspects described below.

FIGS. 1A, 1i, 1C, and 1D show that the NOD2 agonists enhance monocyte FcγR function. FIG. 1A shows that the healthy-donor PBMCs were treated with MDP at 1 μg/mL for 24 hours. Cells were collected, and the indicated markers were evaluated in CD14⁺ cells by flow cytometry (n=3). Top graphs show the expression levels by geometric mean of fluorescence intensity (GMFI); lower panels show representative histograms. FIG. 1B shows that the isolated monocytes were treated with MDP at 1 μg/mL for 24 hours; supernatants were obtained and production of TNFα was quantified by ELISA (n=10). FIGS. 1C and 1D shows that monocytes were treated with MDP at 1 μg/mL for 24 hours. Then, antibody-mediated functions were analyzed: Rosetting (FIG. 1C; n=3) and Phagocytosis (FIG. 1D; n=7). In both cases, the index indicates the number of sRBCs bound/engulfed by 100 monocytes, while the number of active monocytes indicates the percent of active cells (having surface bound or engulfed sRBCs). * p≤0.05, ** p≤0.01, *** p≤0.001, **** p≤0.0001.

FIGS. 2A, 2B, and 2C show that NOD2 stimulation modulates FcγR expression. Monocytes were treated for 24 hours with MDP at 1 μg/mL. Cells were collected to analyze transcripts (FIG. 2A; n=5) and surface expression (FIG. 2B; n=6) of the FcγRs by real-time PCR or flow cytometry, respectively. Bottom histograms show a representative donor. FIG. 2V shows the mRNA and total protein level of the common gamma chain (FcεR1γ) were evaluated through real-time PCR and western blot (n=5). * p≤0.05, ** p≤0.01.

FIGS. 3A, 3B, 3C, 3D, 3E, and 3F show that NF-κB and p38 are required for NOD2-mediated effects on FcγR. Monocytes were treated with inhibitors against NF-κB (top), MEK (middle), or p38 (bottom) for 30 minutes, before stimulation with the MDP for 24 hours. Following incubation, expression of the FcγRs (FIGS. 3A, 3C, and 3E) was measured through flow cytometry (representative histogram of n≥4 is shown); antibody-mediated phagocytosis was evaluated (FIGS. 3, 3D, and 3F; n≥3). *p≤0.05, ** p≤0.01, *** p≤0.001, *** p≤0.0001.

FIGS. 4A, 4B, 4C, 4D, 4E, and 4F show that NOD2 regulates FcγR function in CLL-patient monocytes. FIG. 4A shows the levels of NOD2 expression in healthy-donor and CLL-patient monocytes were measured by real-time PCR (n=3 donors each). CLL-patient monocytes were stimulated for 24 hours with MDP at 1 μg/mL and production of TNFα was evaluated in the supernatant (FIG. 4B; n=8); the levels of FcγRs were measured by flow cytometry (FIG. 4C; n=6) or FcεR1γabundance was observed by western blot (representative blot, FIG. 4D; n=3 donors). FIG. 4E shows antibody-mediated phagocytosis (n=3), or FIG. 4F shows Rosetting (n=4) were also measured. * p≤0.05, ** p≤0.01.

FIGS. 5A, 5B, 5C, 5D, and 5E show NOD2 activation enhances effects of antitumor antibody in vivo. Mice were engrafted with splenocytes from a diseased Eμ-TCL1 mouse and disease allowed to develop for two weeks. Mice were treated with MDP, αCD20 antibody or a combination three times per week for two weeks. Mice were then euthanized, and blood and spleen samples obtained (FIG. 5E). FIG. 5A show the white blood cell counts were taken as explained in the methods. FIG. 5B shows the percent CD19⁺CD5⁺ cells in CD45⁺ peripheral blood was measured by flow cytometry and graphed. FIG. 5C shows the spleen weights were taken, and FIG. 5D shows the number of CD45⁺CD19⁺CD5⁺ cells per milligram spleen weight was measured (n=3 for unengrafted, n=6 for all others). * p≤0.05, * p≤0.05, *** p≤0.001, *** p≤0.0001.

FIGS. 6A, 6B, 6C, and 6D show that the NOD2 agonists enhance antibody-mediated monocyte/macrophage activation in vivo. C57BL/6 mice were engrafted with splenocytes from a diseased Eμ-TCL1 mouse and the disease allowed to develop for two weeks. Mice were treated with MDP, αCD20 antibody or a combination three times a week for two weeks. Then, mice were sacrificed, and spleen samples obtained. Cells were stained for flow cytometry and monocytes/macrophages were identified as CD45⁺CD11b⁺Ly6G⁻ cells and divided by F4/80 and/or MHC-II expression. MHC-II⁺ (FIG. 6A), F4/80⁺ (FIG. 6B), and F4/80⁺MHC-II⁺ (FIG. 6C) cells were then characterized as M1 by iNOS expression, or as M2 by EGR2 expression. Percents of M1 or M2 in the different populations are shown. FIG. 6D shows the representative dot plots from one mouse per group for iNOS/EGR2 expression are shown (n=3 for unengrafted, n=6 for all others). ** p≤0.01, *** p≤0.001, **** p≤0.0001.

FIGS. 7A, 7B, 7C, and 7D show the effects of NOD2 stimulation in other immune-cell populations. Isolated PBMCs were treated with L18-MDP at 1 μg/mL for 24 hours. Then, samples were collected, and the indicated markers were analyzed by flow cytometry in viable CD3⁺CD4⁺ (FIG. 7A), CD3⁺CD8⁺ (FIG. 7B), CD19⁺ (FIG. 7C), and CD56⁺ (FIG. 7D) cells (n=3). * p≤0.05, ** p≤0.01.

FIGS. 8A, 8B and 8C show the verification of inhibitor activity. Monocytes were treated with inhibitors against NF-κB (FIG. 8A), MEK (FIG. 8B), or p38 (FIG. 8C). To ensure appropriate blocking, the activation of downstream targets was assessed by western blot. Figure shows a representative blot for each inhibitor (n≤3). GAPDH was used as loading control.

FIGS. 9A, 9B, and 9C show the inhibition of NF-κB and p38 affects monocyte FcγR transcriptional responses to NOD2. Healthy-donor monocytes were treated with inhibitors for NF-κB (FIG. 9A), MEK (FIG. 9B), and p38 (FIG. 9C) before stimulation with NOD2 agonist for 24 hours. Cells collected, and total RNA obtained. The expression of FcγR was evaluated through real time PCR. * p≤0.05, ** p≤0.01, *** p≤0.001, **** p≤0.0001 (n≤3).

FIGS. 10A, 10B, and 10C show the inhibition of NF-κB and p38 affects NOD2-mediated changes in FcγR surface expression in monocytes. Healthy-donor monocytes were treated with inhibitors for NF-κB (FIG. 10A), MEK (FIG. 10B), and p38 (FIG. 10C) before stimulation with NOD2 agonist for 24 hours. Cells were collected and the expression of FcγR was evaluated through flow cytometry. Expression was calculated by the geometric mean; the percent of FcγRIIIa positive population is also shown. * p≤0.05, ** p≤0.01, *** p≤0.001, **** p≤0.0001 (n≥3).

FIGS. 11A, 11B, and 11C show that MDP stimulation does not induce CLL cell activation and survival in vitro. FIG. 11A shows the PBMCs from CLL patients were isolated and stimulated with 1 μg/mL MDP for 24 hours. Then, cells were collected and the expression of CD86 in B cells was evaluated by flow cytometry (n=3). Isolated B cells from CLL patients were treated once with MDP at 1 μg/mL; then, the number of viable cells (FIG. 11B) and the percent viability (FIG. 11C) was evaluated at the indicated time points (n=3).

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

Reference will now be made in detail to the embodiments of the invention, examples of which are illustrated in the drawings and the examples. This invention may, however, be embodied in many different forms and should not be construed as limited to the embodiments set forth herein.

### Terminology

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification.

The terms “about” and “approximately” are defined as being “close to” as understood by one of ordinary skill in the art. In one non-limiting embodiment the terms are defined to be within 10%. In another non-limiting embodiment, the terms are defined to be within 5%. In still another non-limiting embodiment, the terms are defined to be within 1%.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition, or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, 100% or more increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1% 2% 3% 4% 5% 6% 7% 8% 9% 10% 11% 12% 13% 14% 15% 16% 17% 18% 19% 20% 21% 22% 23% 24% 25% 26% 27% 28% 29% 30% 31% 32% 33% 34% 35% 36% 37% 38% 39% 40% 41% 42% 43% 44% 45% 46% 47% 48% 49% 50% 51% 52% 53% 54% 55% 56% 57% 58% 59% 60% 61% 62% 63% 64% 65% 66% 67% 68% 69% 70% 71% 72% 73% 74% 75% 76% 77% 78% 79% 80% 81% 82% 83% 84% 85% 86% 87% 88% 89% 90% 91% 92% 93% 94% 95% 96% 97% 98% 99% or 100% or more decrease so long as the decrease is statistically significant.

Specifically disclosed herein is a “decrease in tumor burden” which can mean a reduction in tumor size by 1% 2% 3% 4% 5% 6% 7% 8% 9% 10% 11% 12% 13% 14% 15% 16% 17% 18% 19% 20% 21% 22% 23% 24% 25% 26% 27% 28% 29% 30% 31% 32% 33% 34% 35% 36% 37% 38% 39% 40% 41% 42% 43% 44% 45% 46% 47% 48% 49% 50% 51% 52% 53% 54% 55% 56% 57% 58% 59% 60% 61% 62% 63% 64% 65% 66% 67% 68% 69% 70% 71% 72% 73% 74% 75% 76% 77% 78% 79% 80% 81% 82% 83% 84% 85% 86% 87% 88% 89% 90% 91% 92% 93% 94% 95% 96% 97% 98% 99% or 100% compared to the tumor size before treatment. A decrease in tumor burden can also be measured by a decrease in markers associated with the tumor. This decrease in one or more markers can be a decrease of 1% 2% 3% 4% 5% 6% 7% 8% 9% 10% 11% 12% 13% 14% 15% 16% 17% 18% 19% 20% 21% 22% 23% 24% 25% 26% 27% 28% 29% 30% 31% 32% 33% 34% 35% 36% 37% 38% 39% 40% 41% 42% 43% 44% 45% 46% 47% 48% 49% 50% 51% 52% 53% 54% 55% 56% 57% 58% 59% 60% 61% 62% 63% 64% 65% 66% 67% 68% 69% 70% 71% 72% 73% 74% 75% 76% 77% 78% 79% 80% 81% 82% 83% 84% 85% 86% 87% 88% 89% 90% 91% 92% 93% 94% 95% 96% 97% 98% 99% or 100%.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction below, above, or in between the given ranges as compared to native or control levels.

By “reduce” or other forms of the word, such as “reducing” or “reduction,” means lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The terms “treat,” “treating,” and grammatical variations thereof as used herein, include partially or completely delaying, alleviating, mitigating or reducing the intensity of one or more attendant symptoms of a disorder or condition and/or alleviating, mitigating or impeding one or more causes of a disorder or condition. Treatments according to the disclosure may be applied preventively, prophylactically, palliatively or remedially. Treatments are administered to a subject prior to onset (e.g., before obvious signs of tumor growth), during early onset (e.g., upon initial signs and symptoms of cancer), or after an established development of cancer.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective amount” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, a vector, polynucleotide, cells, salts, esters, amides, proagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

The term “administer,” “administering”, or derivatives thereof refer to delivering a composition, substance, inhibitor, or medication to a subject or object by one or more the following routes: oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation or via an implanted reservoir. The term “parenteral” includes subcutaneous, intravenous, intramuscular, intra-articular, intra-synovial, intrasternal, intrathecal, intrahepatic, intralesional, and intracranial injections or infusion techniques.

Reference also is made herein to peptides, polypeptides, proteins, and compositions comprising peptides, polypeptides, and proteins. As used herein, a polypeptide and/or protein is defined as a polymer of amino acids, typically of length≥100 amino acids (Garrett & Grisham, Biochemistry, 2nd edition, 1999, Brooks/Cole, 110). A peptide is defined as a short polymer of amino acids, of a length typically of 20 or less amino acids, and more typically of a length of 12 or less amino acids (Garrett & Grisham, Biochemistry, 2nd edition, 1999, Brooks/Cole, 110). It should be noted that the antibody or monoclonal antibodies of any preceding aspect are proteins and/or compositions comprising polypeptides or proteins.

The peptides, polypeptides, and proteins disclosed herein may be modified to include non-amino acid moieties. Modifications may include but are not limited to carboxylation (e.g., N-terminal carboxylation via addition of a di-carboxylic acid having 4-7 straight-chain or branched carbon atoms, such as glutaric acid, succinic acid, adipic acid, and 4,4-dimethylglutaric acid), amidation (e.g., C-terminal amidation via addition of an amide or substituted amide such as alkylamide or dialkylamide), PEGylation (e.g., N-terminal or C-terminal PEGylation via additional of polyethylene glycol), acylation (e.g., O-acylation (esters), N-acylation (amides), S-acylation (thioesters)), acetylation (e.g., the addition of an acetyl group, either at the N-terminus of the protein or at lysine residues), formylation lipoylation (e.g., attachment of a lipoate, a C8 functional group), myristoylation (e.g., attachment of myristate, a C14 saturated acid), palmitoylation (e.g., attachment of palmitate, a C16 saturated acid), alkylation (e.g., the addition of an alkyl group, such as an methyl at a lysine or arginine residue), isoprenylation or prenylation (e.g., the addition of an isoprenoid group such as farnesol or geranylgeraniol), amidation at C-terminus, glycosylation (e.g., the addition of a glycosyl group to either asparagine, hydroxylysine, serine, or threonine, resulting in a glycoprotein). Distinct from glycation, which is regarded as a nonenzymatic attachment of sugars, polysialylation (e.g., the addition of polysialic acid), glypiation (e.g., glycosylphosphatidylinositol (GPI) anchor formation, hydroxylation, iodination (e.g., of thyroid hormones), and phosphorylation (e.g., the addition of a phosphate group, usually to serine, tyrosine, threonine, or histidine).

The term “antibody” is used in the broadest sense, and specifically covers monoclonal antibodies (including full length monoclonal antibodies), polyclonal antibodies, and multispecific antibodies (e.g., bispecific antibodies). Antibodies (Abs) and immunoglobulins (Igs) are glycoproteins having the same structural characteristics. While antibodies exhibit binding specificity to a specific target, immunoglobulins include both antibodies and other antibody-like molecules which lack target specificity. Native antibodies and immunoglobulins are usually heterotetrameric glycoproteins of about 150,000 daltons, composed of two identical light (L) chains and two identical heavy (H) chains. Each heavy chain has at one end a variable domain (V_{H}) followed by a number of constant domains. Each light chain has a variable domain at one end (V_{L}) and a constant domain at its other end.

The term “antibody fragment” refers to a portion of a full-length antibody, generally the target binding or variable region. Examples of antibody fragments include Fab, Fab′, F(ab′)₂ and Fv fragments. The phrase “functional fragment or analog” of an antibody is a compound having qualitative biological activity in common with a full-length antibody. For example, a functional fragment or analog of an anti-IgE antibody is one which can bind to an IgE immunoglobulin in such a manner so as to prevent or substantially reduce the ability of such molecule from having the ability to bind to the high affinity receptor, FcεRI. As used herein, “functional fragment” with respect to antibodies, refers to Fv, F(ab) and F(ab′)₂ fragments. An “Fv” fragment is the minimum antibody fragment which contains a complete target recognition and binding site. This region consists of a dimer of one heavy and one light chain variable domain in a tight, non-covalent association (V_{H}-V_{L} dimer). It is in this configuration that the three CDRs of each variable domain interact to define a target binding site on the surface of the V_{H}-V_{L} dimer. Collectively, the six CDRs confer target binding specificity to the antibody. However, even a single variable domain (or half of an Fv comprising only three CDRs specific for a target) has the ability to recognize and bind target, although at a lower affinity than the entire binding site. “Single-chain Fv” or “sFv” antibody fragments comprise the V_{H} and V_{L} domains of an antibody, wherein these domains are present in a single polypeptide chain. Generally, the Fv polypeptide further comprises a polypeptide linker between the V_{H} and V_{L} domains which enables the sFv to form the desired structure for target binding.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules.

It should be noted that the terms “variant” and “derivative can be used interchangeably. The terms “variant” or “derivative” mean a polypeptide derived from a parent polypeptide by one or more (several) alteration(s), i.e., a substitution, insertion, and/or deletion, at one or more (several) positions A substitution means a replacement of an amino acid occupying a position with a different amino acid; a deletion means removal of an amino acid occupying a position; and an insertion means adding 1 or more, such as 1, 2, 3, 4, 5, 6, 7, 8, 9 or 10, preferably 1-3 amino acids immediately adjacent an amino acid occupying a position. In relation to substitutions, ‘immediately adjacent’ ray be to the N-side (‘upstream’) or C-side (‘downstream’) of the amino acid occupying a position (‘the named amino acid’). Therefore, for an amino acid named/numbered ‘X,’ the insertion nay be at position ‘X+1’ (‘downstream’) or at position ‘X−1’ (‘upstream’).

A “variant” or “derivative” of a particular polypeptide sequence may be defined as a polypeptide sequence having at least 50% sequence identity to the particular polypeptide sequence over a certain length of one of the polypeptide sequences using blastp with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). In some embodiments a variant polypeptide may show, for example, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length relative to a reference polypeptide.

A variant or derivative polypeptide may have substantially the same functional activity as a reference polypeptide. For example, a variant or derivative polypeptide may exhibit or more biological activities associated with binding a ligand and/or binding DNA at a specific binding site.

A “vaccine” refers to a biological preparation that provides active acquired immunity to a particular infectious diseases caused by a virus, bacteria, parasite, or any other microorganisms. Vaccines typically comprise an agent or several agents, also referred to as antigens, resembling the disease-causing microorganism and is often made from weakened or killed forms of the microbe, its toxins, or its surface proteins/peptides. Vaccines are also made to comprise additional components, such as adjuvants, preservatives, and/or stabilizers to boost the immune response, improve safety, and improve vaccine storage.

The terms “immunotherapy” and “immunotherapeutic” refers to the treatment of disease by activating or suppressing the immune system. In cancer treatment, the most effective immunotherapies are cell-based immunotherapies that utilize lymphocytes, macrophages, dendritic cells, natural killer cells, cytotoxic T lymphocytes, etc. to defend the body against cancer by targeting abnormal antigens expressed on the surface of tumor cells.

The term “variable” in the context of variable domain of antibodies, refers to the fact that certain portions of the variable domains differ extensively in sequence among antibodies and are used in the binding and specificity of each particular antibody for its particular target. However, the variability is not evenly distributed through the variable domains of antibodies. It is concentrated in three segments called complementarity determining regions (CDRs) also known as hypervariable regions both in the light chain and the heavy chain variable domains. The more highly conserved portions of variable domains are called the framework (FR). The variable domains of native heavy and light chains each comprise four FR regions, largely an adopting a .beta.-sheet configuration, connected by three CDRs, which form loops connecting, and in some cases forming part of, the .beta.-sheet structure. The CDRs in each chain are held together in close proximity by the FR regions and, with the CDRs from the other chain, contribute to the formation of the target binding site of antibodies (see Kabat et al.) As used herein, numbering of immunoglobulin amino acid residues is done according to the immunoglobulin amino acid residue numbering system of Kabat et al., (Sequences of Proteins of Immunological Interest, National Institute of Health, Bethesda, Md. 1987), unless otherwise indicated.

An “antigen” refers to a molecule, moiety, foreign particulate matter, or an allergen that can bind to a specific antibody or T cell receptor. The presence of antigens within a host can illicit an immune response against said molecule, moiety, foreign particulate matter, or allergen.

An “adjuvant” refers to a drug, molecule, substance, or a combination thereof that is used to increase the efficacy or potency of certain therapeutic agents, such as for example vaccines and/or antibodies. “Adjuvant(s)” are often at least one ingredient used in some vaccines that help create a stronger immune response in the host receiving said vaccine.

### Immunotherapeutic Compositions

In one aspect, disclosed herein is an immunotherapeutic composition comprising a muramyldipeptide (MDP), a muramyltripeptide (MTP), L18-MDP, M-TriLYS, murabutide, N-Glycolyl-MDP, or a derivative thereof, a monoclonal antibody; and a pharmaceutically acceptable carrier of any preceding aspect.

In some embodiments, the monoclonal antibody comprises an anti-CD20 monoclonal antibody, including but not limited to rituximab, ofatumumab, ocrelizumab, tositumomab, and Obinutuzumab. In some embodiments, the monoclonal antibody comprises an anti-CD52 monoclonal antibody (including, but not limited to alemtuzumab), an anti-CD19 monoclonal antibody (including, but not limited to MOR208, MEDI-551, and XmAb 5871), or a monoclonal antibody that targets an antigen expressed on B/CLL cells.

In some embodiments, the immunotherapeutic composition comprises a vaccine. In some embodiments, the immunotherapeutic composition further comprises an adjuvant, an additive, or a combination thereof. As used herein, an “adjuvant” refers to any material that increases the humoral or cellular immune response to an antigen, such as for example, a cancer cell. The addition of an adjuvant permits the use of a smaller dose of composition components to stimulate an immune response. In some embodiments, the adjuvant includes, but is not limited to metallic oxides (such as for example aluminum hydroxide), alum, lipopolysaccharide (LPS), monophosphoryl A (MPA), CpG nucleic acids, Squalene, AS04 (a combination of MPL and AAHS), AS03 (an oil-in-water emulsion adjuvant that contains α-tocopherol (Vitamin E)), inorganic chelates of salts, gelatins, various paraffin-type oils, synthesized resins, alginates, mucoid, polysaccharide compounds, caseinates, blood-derived substances (such as for example fibrin clots), synthesized oils, petroleum derivatives, saponins, diethyl-aminoethyl (DEAE)-dextran, polyethylene glycol, and polyacrylic acid. In some embodiments, the additive comprises a stabilizer, including but not limited to sugars and gelatin; and a preservative, including but not limited to thimerosal.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

As used herein, the term “carrier” encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia, PA, 2005. Examples of physiologically acceptable carriers include saline, glycerol, DMSO, buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ (ICI, Inc.; Bridgewater, New Jersey), polyethylene glycol (PEG), and PLURONICS™ (BASF; Florham Park, NJ). To provide for the administration of such dosages for the desired therapeutic treatment, compositions disclosed herein can advantageously comprise between about 0.1% and 99% by weight of the total of one or more of the subject compounds based on the weight of the total composition including carrier or diluent.

### Methods of Treating Cancer

The present disclosure provides methods of using immunotherapeutic compositions for treating and/or preventing a cancer, including but not limited to chronic lymphocytic leukemia (CLL). The present disclosure also provides methods of stimulating a monocyte or macrophage using a nucleotide-binding oligomerization domain-containing protein 2 (NOD2) agonist and an immunotherapeutic agent, including but not limited to a monoclonal antibody (mAb).

In one aspect, disclosed herein is a method of treating a cancer, the method comprising administering to a subject a therapeutically effective amount of a composition comprising a nucleotide-binding oligomerization domain-containing protein 2 (NOD2) agonist, an immunotherapeutic agent, and a pharmaceutically acceptable carrier, wherein the method decreases tumor burden of the cancer.

Cancers which can be treated using the disclosed methods include, but are not limited to, acoustic neuroma, adenocarcinoma, adrenal gland cancer, anal cancer, angiosarcoma (e.g., lymphangiosarcoma, lymphangioendotheliosarcoma, hemangiosarcoma), appendix cancer, benign monoclonal gammopathy, biliary cancer (e.g., cholangiocarcinoma), bladder cancer, breast cancer (e.g., adenocarcinoma of the breast, papillary carcinoma of the breast, mammary cancer, medullary carcinoma of the breast), brain cancer (e.g., meningioma; glioma, e.g., astrocytoma, oligodendroglioma; medulloblastoma), bronchus cancer, carcinoid tumor, cervical cancer (e.g., cervical adenocarcinoma), choriocarcinoma, chordoma, craniopharyngioma, colorectal cancer (e.g., colon cancer, rectal cancer, colorectal adenocarcinoma), epithelial carcinoma, ependymoma, endotheliosarcoma (e.g., Kaposi's sarcoma, multiple idiopathic hemorrhagic sarcoma), endometrial cancer (e.g., uterine cancer, uterine sarcoma), esophageal cancer (e.g., adenocarcinoma of the esophagus, Barrett's adenocarcinoma), Ewing's sarcoma, eye cancer (e.g., intraocular melanoma, retinoblastoma), familiar hypereosinophilia, gall bladder cancer, gastric cancer (e.g., stomach adenocarcinoma), gastrointestinal stromal tumor (GIST), head and neck cancer (e.g., head and neck squamous cell carcinoma, oral cancer (e.g., oral squamous cell carcinoma (OSCC), throat cancer (e.g., laryngeal cancer, pharyngeal cancer, nasopharyngeal cancer, oropharyngeal cancer)), hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CVL) (e.g., B-cell CML, T-cell CML), and chronic lymphocytic leukemia (CLL) (e.g., B-cell CLL, T-cell CLL); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma (DLBCL)), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., “Waldenstrom's macroglobulinemia”), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma); a mixture of one or more leukemia/lymphoma as described above; and multiple myeloma (MM)), heavy chain disease (e.g., alpha chain disease, gamma chain disease, mu chain disease), hemangioblastoma, inflammatory myofibroblastic tumors, immunocytic amyloidosis, kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), leiomyosarcoma (LMS), mastocytosis (e.g., systemic mastocytosis), myelodysplastic syndrome (MDS), mesothelioma, myeloproliferative disorder (MPD) (e.g., polycythemia Vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)), neuroblastoma, neurofibroma (e.g., neurofibromatosis (NF) type 1 or type 2, schwannomatosis), neuroendocrine cancer (e.g., gastroenteropancreatic neuroendoctrine tumor (GEP-NET), carcinoid tumor), osteosarcoma, ovarian cancer (e.g., cystadenocarcinoma, ovarian embryonal carcinoma, ovarian adenocarcinoma), papillary adenocarcinoma, pancreatic cancer (e.g., pancreatic adenocarcinoma, intraductal papillary mucinous neoplasm (IPMN), Islet cell tumors), penile cancer (e.g., Paget's disease of the penis and scrotum), pinealoma, primitive neuroectodermal tumor (PNT), prostate cancer (e.g., prostate adenocarcinoma), rectal cancer, rhabdomyosarcoma, salivary gland cancer, skin cancer (e.g., squamous cell carcinoma (SCC), keratoacanthoma (KA), melanoma, basal cell carcinoma (BCC)), small bowel cancer (e.g., appendix cancer), soft tissue sarcoma (e.g., malignant fibrous histiocytoma (MFH), liposarcoma, malignant peripheral nerve sheath tumor (MPNST), chondrosarcoma, fibrosarcoma, myxosarcoma), sebaceous gland carcinoma, sweat gland carcinoma, synovioma, testicular cancer (e.g., seminoma, testicular embryonal carcinoma), thyroid cancer (e.g., papillary carcinoma of the thyroid, papillary thyroid carcinoma (PTC), medullary thyroid cancer), urethral cancer, vaginal cancer and vulvar cancer (e.g., Paget's disease of the vulva).

In one aspect, disclosed herein is a method of treating a chronic lymphocytic leukemia (CLL), the method comprising administering to a subject in need thereof a therapeutically effective amount of a nucleotide-binding oligomerization domain-containing protein 2 (NOD2) agonist and an immunotherapeutic agent, wherein the method decreases tumor burden of CLL relative to an untreated control.

In one aspect, disclosed herein is a method of treating a chronic lymphocytic leukemia (CCL), the method comprising administering to a subject a therapeutically effective amount of a composition comprising a nucleotide-binding oligomerization domain-containing protein 2 (NOD2) agonist, an immunotherapeutic agent, and a pharmaceutically acceptable carrier, wherein the method decreases tumor burden of CLL.

An “agonist” refers to a chemical composition or compound that activates a receptor protein to produce a biological response. In some embodiments, the NOD2 agonist comprises a muramyldipeptide (MDP), a muramyltripeptide (MTP), L18-MDP, M-TriLYS, murabutide, N-Glycolyl-MDP, or a derivative thereof. In some embodiments, the immunotherapeutic agent comprises a monoclonal antibody of any preceding aspect. In some embodiments, the monoclonal antibody binds to a tumor cell and interacts with a constant fragment (Fc) on an effector cell. As used herein, an “effector cell” is any type of cell that actively responds to a stimulus and effects some change to itself, other cells, or its environment. Examples of effector cells include, but are not limited to B cells, T cells, natural killer (NK) cells, monocytes, and macrophages.

The immunotherapeutic composition of any preceding aspect may be administered in such amounts, time, and route deemed necessary in order to achieve the desired result. The exact amount of the immunotherapeutic composition of any preceding aspect will vary from subject to subject, depending on the species, age, and general condition of the subject, the severity of the disease, the particular immunotherapeutic composition of any preceding aspect, its mode of administration, its mode of activity, and the like. The immunotherapeutic composition of any preceding aspect is preferably formulated in dosage unit form for ease of administration and uniformity of dosage. It will be understood, however, that the total daily usage of the immunotherapeutic composition of any preceding aspect will be decided by the attending physician within the scope of sound medical judgment. The specific therapeutically effective dose level for any particular subject will depend upon a variety of factors including the disease being treated and the severity of the disease symptoms; the activity of the immunotherapeutic composition of any preceding aspect employed; the specific immunotherapeutic composition of any preceding aspect employed; the age, body weight, general health, sex and diet of the patient; the time of administration, route of administration, and rate of excretion of the specific immunotherapeutic composition of any preceding aspect employed; the duration of the treatment; drugs used in combination or coincidental with the specific immunotherapeutic composition of any preceding aspect employed; and like factors well known in the medical arts.

The immunotherapeutic composition of any preceding aspect may be administered by any route. In some embodiments, the immunotherapeutic composition of any preceding aspect is administered via a variety of routes, including oral, intravenous, intramuscular, intra-arterial, intramedullary, intrathecal, subcutaneous, intraventricular, transdermal, interdermal, rectal, intravaginal, intraperitoneal, mucosal, nasal, buccal, enteral, sublingual; by intratracheal instillation, bronchial instillation, and/or inhalation. In general, the most appropriate route of administration will depend upon a variety of factors including the nature of the immunotherapeutic composition of any preceding aspect (e.g., its stability in the environment of the subject's body), the condition of the subject (e.g., whether the subject is able to tolerate the chosen route of administration), etc.

The exact amount of immunotherapeutic composition of any preceding aspect required to achieve a therapeutically effective amount will vary from subject to subject, depending on species, age, and general condition of a subject, severity of the side effects, identity of the particular compound(s), mode of administration, and the like. The amount to be administered to, for example, a child or an adolescent can be determined by a medical practitioner or person skilled in the art and can be lower or the same as that administered to an adult.

In some embodiments, the immunotherapeutic composition of any preceding aspect can be prepared as a “concentrate”, e.g. in a storage container of a premeasure volume and/or a predetermined amount ready for dilution, or in a prediluted composition that is ready for administration.

In some embodiments, the immunotherapeutic composition of any preceding aspect is administered 1, 2, 3, 4, 5, or more times. In some embodiments, the immunotherapeutic composition of any preceding aspect is administered daily. In some embodiments, the immunotherapeutic composition of any preceding aspect is administered every day, every 2 days, every 3 days, every 4 days, every 5 days, every 6 days, every 7 days, or more. In some embodiments, the immunotherapeutic composition of any preceding aspect is administered every week, every 2 weeks, every 3 weeks, every 4 weeks, or more. In some embodiments, the immunotherapeutic composition of any preceding aspect is administered every month, every 2 months, every 3 months, every 4 months, every 5 months, every 6 months, every 7 months, every 8 months, every 9 months, every 10 months, every 11 months, every 12 months, or more. In some embodiments, the immunotherapeutic composition of any preceding aspect is administered every year, every 2 years, every 3 years, every 4 years, every 5 years, or more.

In some embodiments, the NOD2 agonist and the immunotherapeutic agent are administered concurrently. In some embodiments, the NOD2 agonist and the immunotherapeutic agent are administered consecutively. In some embodiments, the method further comprises administering an adjuvant, an additive, or a combination thereof of any preceding aspect. In some embodiments, the adjuvant, additive, or a combination are administered concurrently or consecutively with the NOD2 agonist and the immunotherapeutic agent. In some embodiments, the method comprises a pharmaceutically acceptable carrier of any preceding aspect.

In some embodiments, the NOD2 agonist of any preceding aspect is administered at a non-limiting dose of 0.05-10 mg/m². In some embodiments, the NOD2 agonist of any preceding aspect is administered at a non-limiting dose of 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.05, 1.1, 1.15, 1.2, 1.25, 1.3, 1.35, 1.4, 1.45, 1.5, 1.55, 1.6, 1.65, 1.7, 1.75, 1.8, 1.85, 1.9, 1.95, 2, 2.05, 2.1, 2.15, 2.2, 2.25, 2.3, 2.35, 2.4, 2.45, 2.5, 2.55, 2.6, 2.65, 2.7, 2.75, 2.8, 2.85, 2.9, 2.95, 3, 3.05, 3.1, 3.15, 3.2, 3.25, 3.3, 3.35, 3.4, 3.45, 3.5, 3.55, 3.6, 3.65, 3.7, 3.75, 3.8, 3.85, 3.9, 3.95, 4, 4.05, 4.1, 4.15, 4.2, 4.25, 4.3, 4.35, 4.4, 4.45, 4.5, 4.55, 4.6, 4.65, 4.7, 4.75, 4.8, 4.85, 4.9, 4.95, 5, 5.05, 5.1, 5.15, 5.2, 5.25, 5.3, 5.35, 5.4, 5.45, 5.5, 5.55, 5.6, 5.65, 5.7, 5.75, 5.8, 5.85, 5.9, 5.95, 6, 6.05, 6.1, 6.15, 6.2, 6.25, 6.3, 6.35, 6.4, 6.45, 6.5, 6.55, 6.6, 6.65, 6.7, 6.75, 6.8, 6.85, 6.9, 6.95, 7, 7.05, 7.1, 7.15, 7.2, 7.25, 7.3, 7.35, 7.4, 7.45, 7.5, 7.55, 7.6, 7.65, 7.7, 7.75, 7.8, 7.85, 7.9, 7.95, 8, 8.05, 8.1, 8.15, 8.2, 8.25, 8.3, 8.35, 8.4, 8.45, 8.5, 8.55, 8.6, 8.65, 8.7, 8.75, 8.8, 8.85, 8.9, 8.95, 9, 9.05, 9.1, 9.15, 9.2, 9.25, 9.3, 9.35, 9.4, 9.45, 9.5, 9.55, 9.6, 9.65, 9.7, 9.75, 9.8, 9.85, 9.9, 9.95, or 10 mg/m². In some embodiments, the NOD2 agonist of any preceding aspect is administered at a non-limiting dose of 0.05-6 mg/m² (PMID: 2787207).

In some embodiments, the method further comprises administering an anti-cancer agent including, but not limited to interferons, cytokines (e.g., tumor necrosis factor, interferon α, interferon γ), vaccines, hematopoietic growth factors, monoclonal serotherapy, immunostimulants and/or immunomodulatory agents (e.g., IL-1, 2, 4, 6, or 12), immune cell growth factors (e.g., GM-CSF) and antibodies (e.g. HERCEPTIN (trastuzumab), T-DM1, AVASTIN (bevacizumab), ERBITUX (cetuximab), VECTIBIX (panitumumab), RITUXAN (rituximab), BEXXAR (tositumomab)).

In some embodiments, the method further comprises administering a chemotherapeutic agent including, but are not limited to, anti-estrogens (e.g. tamoxifen, raloxifene, and megestrol), LHRH agonists (e.g. goserelin and leuprolide), anti-androgens (e.g. flutamide and bicalutamide), photodynamic therapies (e.g. verteporfin (BPD-MA), phthalocyanine, photosensitizer Pc4, and demethoxy-hypocrellin A (2BA-2-DMHA)), nitrogen mustards (e.g. cyclophosphamide, ifosfamide, trofosfamide, chlorambucil, estramustine, and melphalan), nitrosoureas (e.g. carmustine (BCNU) and lomustine (CCNU)), alkylsulphonates (e.g. busulfan and treosulfan), triazenes (e.g. dacarbazine, temozolomide), platinum containing compounds (e.g. cisplatin, carboplatin, oxaliplatin), vinca alkaloids (e.g. vincristine, vinblastine, vindesine, and vinorelbine), taxoids (e.g. paclitaxel or a paclitaxel equivalent such as nanoparticle albumin-bound paclitaxel (ABRAXANE), docosahexaenoic acid bound-paclitaxel (DHA-paclitaxel, Taxoprexin), polyglutamate bound-paclitaxel (PG-paclitaxel, paclitaxel poliglumex, CT-2103, XYOTAX), the tumor-activated prodrug (TAP) ANG1005 (Angiopep-2 bound to three molecules of paclitaxel), paclitaxel-EC-1 (paclitaxel bound to the erbB2-recognizing peptide EC-1), and glucose-conjugated paclitaxel, e.g., 2′-paclitaxel methyl 2-glucopyranosyl succinate; docetaxel, taxol), epipodophyllins (e.g. etoposide, etoposide phosphate, teniposide, topotecan, 9-aminocamptothecin, camptoirinotecan, irinotecan, crisnatol, mytomycin C), anti-metabolites, DIFR inhibitors (e.g. methotrexate, dichloromethotrexate, trimetrexate, edatrexate), IMP dehydrogenase inhibitors (e.g. mycophenolic acid, tiazofurin, ribavirin, and EICAR), ribonucleotide reductase inhibitors (e.g. hydroxyurea and deferoxamine), uracil analogs (e.g. 5-fluorouracil (5-FU), floxuridine, doxifluridine, ratitrexed, tegafur-uracil, capecitabine), cytosine analogs (e.g. cytarabine (ara C), cytosine arabinoside, and fludarabine), purine analogs (e.g. mercaptopurine and Thioguanine), Vitamin D3 analogs (e.g. EB 1089, CB 1093, and KH 1060), isoprenylation inhibitors (e.g. lovastatin), dopaminergic neurotoxins (e.g. 1-methyl-4-phenylpyridinium ion), cell cycle inhibitors (e.g. staurosporine), actinomycin (e.g. actinomycin D, dactinomycin), bleomycin (e.g. bleomycin A2, bleomycin B2, peplomycin), anthracycline (e.g. daunorubicin, doxorubicin, pegylated liposomal doxorubicin, idarubicin, epirubicin, pirarubicin, zorubicin, mitoxantrone), MDR inhibitors (e.g. verapamil), Ca²⁺ ATPase inhibitors (e.g. thapsigargin), imatinib, thalidomide, lenalidomide, tyrosine kinase inhibitors (e.g., axitinib (AG013736), bosutinib (SKI-606), cediranib (RECENTIN™, AZD2171), dasatinib (SPRYCEL, BMS-354825), erlotinib (TARCEVA), gefitinib (IRESSA), imatinib (Gleevec, CGP57148B, STI-571), lapatinib (TYKERB, TYVERB), lestaurtinib (CEP-701), neratinib (HKI-272), nilotinib (TASIGNA), semaxanib (semaxinib, SU5416), sunitinib (SUTENT, SU11248), toceranib (PALLADIA), vandetanib (ZACTIMA, ZD6474), vatalanib (PTK787, PTK/ZK), trastuzumab (HERCEPTIN), bevacizumab (AVASTIN), rituximab (RITUXAN), cetuximab (ERBITUX), panitumumab (VECTIBIX), ranibizumab (Lucentis), nilotinib (TASIGNA), sorafenib (NEXAVAR), everolimus (AFINITOR), alemtuzumab (CAMPATH), gemtuzumab ozogamicin (MYLOTARG), temsirolimus (TORISEL), ENMD-2076, PCI-32765, AC220, dovitinib lactate (TK1258, CHIR-258), BIBW 2992 (TOVOK™), SGX523, PF-04217903, PF-02341066, PF-299804, BMS-777607, ABT-869, MP470, BIBF 1120 (VARGATEF), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (VELCADE)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genetech), SF1126 (Semafoe) and OSI-027 (OSI)), oblimersen, gemcitabine, caminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, caminomycin, aminopterin, and hexamethyl melamine.

In some embodiments, the NOD2 agonist and the immunotherapeutic agent increases an antibody-mediated response relative to the immunotherapeutic agent alone. In some embodiments, the NOD2 agonist and the immunotherapeutic agent increases the antibody-mediated response by 10%, 15%, 20%, 25%, 30%, 35%, 40%, 45%, 50%, 55%, 60%, 65%, 70%, 75%, 80%, 85%, 90%, 95%, 99%, 100%, or more relative to the immunotherapeutic agent alone. In some embodiments, the antibody-mediated response comprises monocyte or macrophage activation. In some embodiments, the method shifts a monocyte or a macrophage from an M2-like phenotype to an M1-like phenotype.

### Methods of Stimulating Immune Cells

Monocytes are a type of white blood cell that undergo a process called “macrophage conversion or reprogramming”, wherein they differentiate into macrophages once they leave the bloodstream and enter into a tissue or organ. Typically, monocytes circulate in the blood for one to three days before migrating into tissues where they become macrophages. Once in the macrophage state, macrophages can be either an M1 macrophage or a M2 macrophage. An M1 macrophage is characterized by pro-inflammatory responses and catabolic processes. An M2 macrophage is characterized by anti-inflammatory responses and anabolic processes. M1 to M2 macrophage conversion is tightly regulated by signaling pathways, transcriptional and posttranscriptional networks. Such conversions are also associated with tumor progression wherein protective M1 macrophages convert to pro-tumorigenic M2 macrophages. Alternatively, M2 to M1 macrophage conversions restrict tumor progression. Thus, the present disclosure provides therapeutic interventions to stimulate monocytes or macrophages to promote the shift from an M2-like macrophage to an M1-like macrophage.

In one aspect, disclosed herein is a method of stimulating a monocyte or a macrophage, the method comprising exposing the monocyte or the macrophage to an effective amount of a nucleotide-binding oligomerization domain-containing protein 2 (NOD2) agonist and an immunotherapeutic agent, wherein the method shifts the monocyte or the macrophage from an M2-like phenotype to an M1-like phenotype.

In one aspect, disclosed herein is a method of treating a subject in need thereof, the method comprising stimulating a monocyte of macrophage by exposing the monocyte or macrophage to an effective amount of a NOD2 agonist and an immunotherapeutic agent, wherein the method shifts the monocyte or the macrophage from the M2-like phenotype to an M1-like phenotype, and wherein the monocyte or macrophage is administered to the subject after being stimulated. In some embodiments, the subject has a cancer of any preceding aspect.

In some embodiments, the NOD2 agonist comprises a muramyldipeptide (MDP), a muramyltripeptide (MTP), L18-MDP, M-TriLYS, murabutide, N-Glycolyl-MDP, or a derivative thereof. In some embodiments, the immunotherapeutic agent comprises a monoclonal antibody of any preceding aspect. In some embodiments, the monoclonal antibody binds to a tumor cell and interacts with a constant fragment (Fc) on an effector cell of any preceding aspect.

In some embodiments, the NOD2 agonist and the immunotherapeutic agent are administered according to any preceding aspect. In some embodiments, the NOD2 agonist and the immunotherapeutic agent are administered concurrently. In some embodiments, the NOD2 agonist and the immunotherapeutic agent are administered consecutively. In some embodiments, the method of stimulating a monocyte or a macrophage further comprises administering an adjuvant, an additive, or a combination thereof. In some embodiments, the adjuvant, additive, or a combination are administered concurrently or consecutively with the NOD2 agonist and the immunotherapeutic agent of any preceding aspect. In some embodiments, the pharmaceutically acceptable carrier of any preceding aspect.

In some embodiments, the NOD2 agonist and the immunotherapeutic agent increases an antibody-mediated response relative to the immunotherapeutic agent alone. In some embodiments, the NOD2 agonist and the immunotherapeutic agent increases the antibody-mediated response by 10%, 15%, 20%, 25%, 30%, 35%, 40%, 45%, 50%, 55%, 60%, 65%, 70%, 75%, 80%, 85%, 90%, 95%, 99%, 100%, or more relative to the immunotherapeutic agent alone. In some embodiments, the antibody-mediated response comprises monocyte or macrophage activation.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

The following examples are set forth below to illustrate the compositions, devices, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1: Methods of Enhancing Antibody Therapies Using NOD2 Agonists

Chronic lymphocytic leukemia (CLL) involves the accumulation of mature B cells in peripheral blood and lymphoid organs. An estimated 20,160 new cases of CLL and 4,410 deaths were expected for 2022. Monoclonal antibody therapies with anti-CD20 antibodies, such as rituximab promote clearance of malignant B cells. These antibodies bind to Fc gamma receptors (FcgR), leading to responses such as phagocytosis and inflammatory cytokine release. However, monocytes/macrophages are suppressed in CLL. Some also differentiate into nurse like cells (NLCs), which are primed by CLL cells to perform supportive, tumorigenic functions. Activation of Toll-like receptors (TLRs) has improved the efficacy of antibody therapies in murine models. Indeed many TLR ligands are currently in clinical trials for various types of cancer. Modeling off this, nucleotide-binding oligomerization domain-containing protein 2 (NOD2) was investigated in this capacity, as the NOD2 agonist muramyl tripeptide phosphatidylethanolamine (MTP-PE) is used for treatment of osteosarcoma.

NOD2, an intracellular pattern recognition receptor, is activated by muramyl dipeptide (MDP) (found in the cell walls of G⁺ and G⁻ bacteria). NOD2-ligand binding leads to downstream activation of NF-kB, the MAPK pathway, and caspase-1. This ultimately leads to production of multiple proinflammatory cytokines, such as IL-1b. Thus, it was contemplated that NOD2 stimulation would enhance antibody-mediated responses in monocytes and alter NLC polarization in CLL.

Monocytes from healthy donors and CLL patients were first used to investigate the effect of NOD2 on the FcgRs. Using qPCR analysis, a downregulation of the inhibitory FcgRIIb was detected following NOD2 stimulation in healthy-donor monocytes and CLL monocytes/NLCs. There was also an upregulation of the activating FcgRIa and FcgRIIIa, as well as the associated g-chain through flow cytometry and western blot analyses, respectively. Additionally, the effect of stimulation on CLL monocyte/macrophage polarization, identifying a decrease in the tumorigenic M2 marker CD163 and an increase in the M1 inflammatory marker CD86 was analyzed. Consistent with the aforementioned results, an increase in TNF-α secretion in healthy donor and CLL monocytes, following stimulation with L18-MDP then antibody, in vitro, was discovered. Furthermore, FcgR-mediated phagocytosis was greatly enhanced following NOD2 stimulation. This was then held in vivo, where combining NOD2 agonists with anti-CD20 antibody resulted in a significant decrease in disease burden in a mouse model of CLL, contrasting with responses from either treatment alone. In conclusion, this shows that NOD2 agonists can be considered as an immunostimulatory agent for use in combination with monoclonal antibody therapies for CLL.

### Example 2: NOD2 Activation Enhances Macrophage Fcγ Receptor Function to Increase the Efficacy of Antibody Therapy

Antibody therapy has been successfully used to treat oncologic disease for more than 20 years. Rituximab, the first therapeutically available antibody, is directed against CD20, expressed on B cells. Upon treatment, B cells are directly killed by antibody binding, by complement and by engagement of immune effector cells. For effective elimination of the target, the activation of cells capable of effecting antibody-mediated responses is crucially needed, including both myeloid cells and natural killer (NK) cells. Due to its importance, novel therapeutic antibodies have been widely engineered to enhance the binding to Fc γ receptors (FcγRs) and thus activate cells in a more efficient manner.

It is known that innate immune responses are broadly suppressed in cancer. In chronic lymphocytic leukemia (CLL), myeloid cells express higher amounts of regulatory molecules and show decreased expression of some molecules related to antibody-mediated phagocytosis, which impedes proper activation upon antigen binding. More importantly, monocytes can differentiate into nurse-like cells (NLCs), which are macrophages widely known to support the survival and drug resistance of CLL cells. Thus, addressing the suppressive phenotype of myeloid cells is crucial to elicit better antibody-mediated responses in CLL.

To increase their anti-tumoral activity, monocytes/macrophages can be stimulated by various agonists of pattern recognition receptors (PPRs). The TLR7 agonist Imiquimod has been successfully used to induce myeloid cell activity in melanoma. The TLR8 agonist Motolimod has been tested against head and neck squamous cell carcinoma in mice and humans, and is showing preclinical efficacy against AML. Many other PRR agonists are currently being examined, while only three of them (BCG, monophosphoryl lipid A, and Imiquimod) are currently used against oncologic diseases.

The potential benefits of treating CLL with PRR agonists have been previously explored. Of note, several reports support an effect of TLR9 agonists towards CLL-cell phenotypic change, increased cytokine production, and apoptosis. However, CpG 7909 in a phase I clinical trial did not result in a clear response and testing of CpG 2006 showed that it may halt T-cell proliferation. Thus, further exploration of PRRs for CLL is needed.

In addition to cell-surface and endosomal PRRs, there are also intracellular sensors that help detect threats inside the cell. Such is the case of nucleotide-binding oligomerization domains 1 and 2 (NOD1 and NOD2), receptors that recognizes peptidoglycans found in both Gram-negative and Gram-positive bacteria. Muramyl-dipeptide has been found to be the minimal unit that can activate NOD2, enhancing the production of humoral responses. Upon activation, NOD2 activates different signaling cascades including the inflammasome, NF-κB and MAPK pathways, leading to a variety of responses including cytokine production. In myeloid cells, activation of NOD2 has been linked to the production of TNFα, IL-6 and/or IL-1β, increased cytolytic activity against tumoral cells, enhanced production of reactive oxygen species and differentiation from classical to non-classical monocytes. The NOD2 agonist MTP-PE (Mifamurtide) has been used therapeutically to treat osteosarcoma patients in Europe, while other agonists have been used for different diseases in other parts of the world. This supports NOD2 agonists as therapeutic adjuvants to enhance responses in myeloid cells. These agonists have also been explored for the treatment of other malignancies including acute myeloid leukemia.

Herein, the role of NOD2 activation was investigated in monocyte/macrophage antibody-mediated responses against CLL. The NOD2 agonist L18-MDP activated CLL-patient monocytes, promoting the production of cytokines and an increase in the expression of FcγRs both at the transcript and protein levels. This was highly correlated to an increase in antibody-mediated phagocytosis. These effects were mediated by the NF-κB and MAPK/p38 pathways. Furthermore, NOD2 agonist treatment led to a decreased leukemic load and a shift in the phenotype of monocytes/macrophages within the Eμ-TCL1 mouse model of CLL. Finally, the agonist significantly enhanced the effects of antitumor antibody. Collectively, these results show that NOD2 agonists can be used as adjuvants for antibody therapy in CLL.

### Methods

Cell culture. Healthy-donor (HD) source leukocytes were purchased from Versiti Blood Services (Columbus, OH, USA). Whole-blood samples from untreated, low-count (<60,000 cells/μl) CLL patients were provided by the Leukemia Tissue Bank of The Ohio State University Comprehensive Cancer Center, obtained according to the Declaration of Helsinki and under protocols approved by the institutional review board at The Ohio State University. Peripheral blood mononuclear cells (PBMCs) were isolated from each sample using lymphocyte separation medium (Corning, Corning, NY, USA), density gradient centrifugation, and then washed with RPMI (Gibco, Thermo-Fisher Scientific, Waltham, MA, USA). For PBMC experiments, cells were counted using the Cellometer Auto 2000 (Nexcelom Biosciences, Lawrence, MA, USA) and resuspended in RPMI media supplemented with 10% FBS (VWR, Radnor, PA, USA), 2 mM L-glutamine (Gibco) and 100 U/mL Penicillin-Streptomycin (Gibco) at 10×10⁶ cells/mL.

To obtain HD or CLL-patient monocytes (CD14⁺ cells), as well as CLL cells (CD19⁺), PBMCs were incubated with magnetic CD14⁺ or CD19⁺ selection beads (Miltenyi Biotec, Waltham, MA, USA) for 15 minutes on ice. Following this, cells were centrifuged and resuspended in MACS buffer (Miltenyi Biotec) and added to an LS selection column. After three washes, cells were recovered from the column, washed with incomplete RPMI, counted, and resuspended at 3×10⁶ cells/mL in supplemented RPMI media.

For stimulation, cells were plated and treated with L18-MDP (MDP)(InvivoGen, San Diego, CA, USA) at 1 μg/mL. Cells were incubated at 37° C., 5% CO₂, for 24 hours unless indicated otherwise. For inhibition experiments, monocytes were treated with Bay 11-7085 at 5 μM (Millipore Sigma, St. Louis, MO, USA), trametinib at 10 nM (Selleck Chemicals, Houston, TX, USA), or SB202190 at 1 μM (Selleck Chemicals) for 30 minutes before MDP stimulation. Dimethyl sulfoxide (DMSO; Sigma Aldrich, St. Louis, MO, USA) was used to prepare stock solutions and the final concentration of DMSO was under 1 L/mL.

Quantitative real-time PCR (qRT-PCR). Cultured cells were collected and then mRNA extracted using the Norgen Total RNA extraction kit (Norgen Biotek, ON, Canada), according to the manufacturer's instructions. Collected mRNA was quantified using the nanodrop ND100 (Thermo-Fisher Scientific), and then reverse transcribed using the high-capacity reverse transcriptase kit (Applied Biosystems, Thermo-Fisher Scientific) into cDNA. Then, qualitative real-time PCR was done using SYBR green (Applied Biosystems) and the QuantStudio 3 machine (Life Technologies). RCN (relative copy number) values were calculated for each target, normalizing to a GAPDH housekeeping control according to the equation RCN=2^{(-ΔCt)}×100, where ΔCt=CT_{problem}−CT_{GAPDH}). Primers used are shown in Table 1.

Immunoblot analysis. Cells were collected and lysed using 70 μL TN1 lysis buffer for every 1×10⁶ cells, as previously described. Protein was quantified using the DC Protein Assay according to manufacturer's instructions (Bio-Rad, Hercules, CA, USA). Samples were boiled with 5× SDS sample buffer (150 mM Tris; 11.5% SDS; 0.05% bromophenol blue; 50% glycerol; 1% 2-mercaptoethanol). Samples were then size-separated using SDS-PAGE. Proteins were transferred to a nitrocellulose membrane using the Trans-Blot Turbo Transfer System (Bio-Rad). Membranes were blocked with the LI-COR Blocking solution (LI-COR Biotechnology, NE, USA) and incubated with primary antibodies overnight at 4° C. in LI-COR antibody diluent. The following primary antibodies were utilized: phospho-p42/44 (polyclonal, product #9101; or clone D13.14.4E; Cell Signaling Technology), phospho-MAPKAPK2 (polyclonal, product #3041; Cell Signaling Technology), phospho-p38 (clone D3F9; Cell Signaling Technology), phospho-p65 (clone 93H1; Cell Signaling Technology) mouse anti-human GAPDH (clone D4C6R; Cell Signaling Technology), rabbit anti-human FcεRIγ (product 06-727; Upstate, Sigma-Aldrich), and mouse anti-human calreticulin (clone FMC 75, Enzo Life Sciences, Farmingdale, NY, USA). After probing with the corresponding fluorescently-conjugated secondary antibody (IRDye 800CW goat anti-rabbit, or IRDye 680RD goat anti-mouse antibodies; LI-COR) for 1 hour at room temperature, membranes were developed using the Odyssey CLx (LI-COR). For densitometry analysis, Fiji was used.

Flow cytometry. After PBMC or monocyte stimulation, cells were collected and washed with PBS. Then, cells were transferred into v-bottom plates and incubated with whole human IgG (Jackson ImmunoResearch Labs, West Grove, PA, USA) at 10 mg/mL in PBS for 15 minutes on ice. After Fc block, the specific antibody cocktail was added (Table 2). Samples were acquired using the LSR-Fortessa (BD Biosciences) at the Flow Cytometry Shared Resource at The Ohio State University Comprehensive Cancer Center and analyzed using FlowJo version 10.7.2 (Ashland, OR, USA).

ELISA. Cultured cells were centrifuged, and cleared supernatants collected and used for ELISA with the Human TNFα DuoSet ELISA kit (R&D Systems, Minneapolis, MN) according to manufacturer's instructions. For acquisition, the BioTek Synergy HTX plate reader was used (Agilent, Santa Clara, CA, USA).

Antibody-mediated binding (roseeting). To determine antibody-mediated binding of targets, opsonized sheep red blood cells (sRBCs; Colorado Serum Company, Denver, CO) were used as previously described (41). sRBCs where washed with PBS until the supernatant appeared clear. Then, 2 μL of PKH26 Red Fluorescent Dye (Sigma-Aldrich), diluted in Diluent C were added to the cell pellet and thoroughly mixed. The reaction was stopped with a 1:1 volume of FBS, and sRBCs were washed with PBS until clear. Fluorescent sRBCs were then opsonized with goat-anti sheep antibody (Sigma-Aldrich) on ice for 2 hours before washing.

Upon monocyte stimulation with MDP at 1 μg/mL for 24 hours, cells were used for rosetting analysis. Briefly, monocytes were divided and incubated with 1 uL of packed opsonized, fluorescent sRBCs. Cells were mixed and spun down, then incubated on ice for 1 hour. After incubation, cells were fixed with 1% paraformaldehyde in PBS for 15 minutes before analysis. The number of bound targets per 100 monocytes were counted through microscopy using the Olympus BX41 (Olympus Lide-Science/Evident, Tokyo, Japan). Samples were counted in a blinded fashion by two different subjects. The number of bound non-opsonized targets in control samples was subtracted from the number of bound opsonized targets.

Antibody-mediated phagocytosis. sRBCs were stained with the pH sensitive Protonex Red 600 (AAT Bioquest, Pleasanton, CA) dye at 4 μM in Hanks Buffered Saline Solution (HBSS, Gibco). The staining was done for 30 minutes at 37° C. and then sRBCs were washed 3 times with PBS. sRBCs were then opsonized with antibody as stated above.

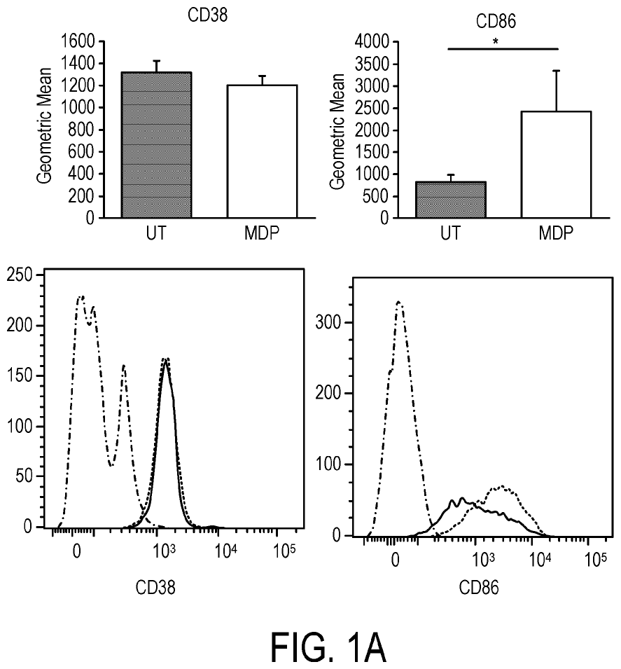

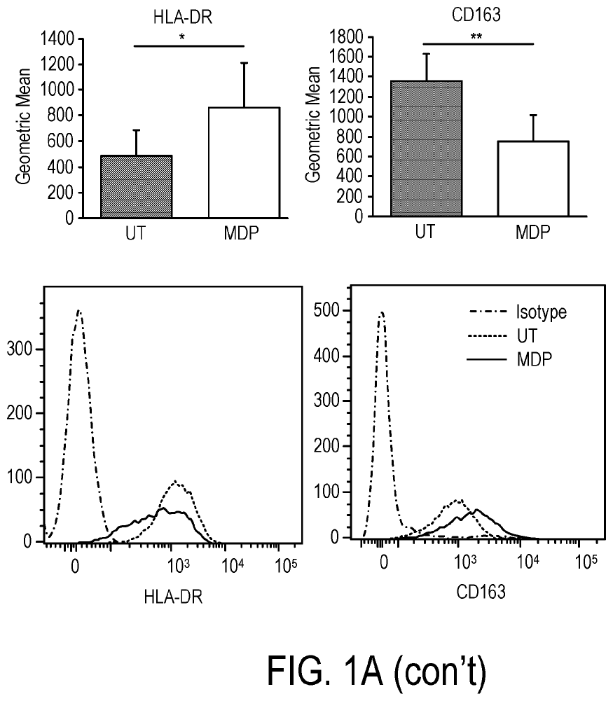

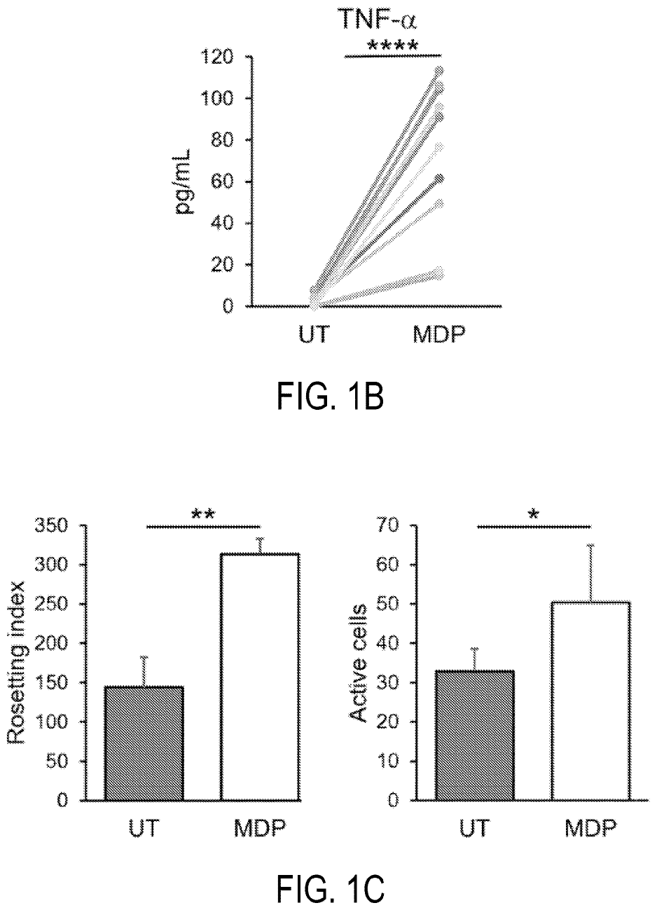

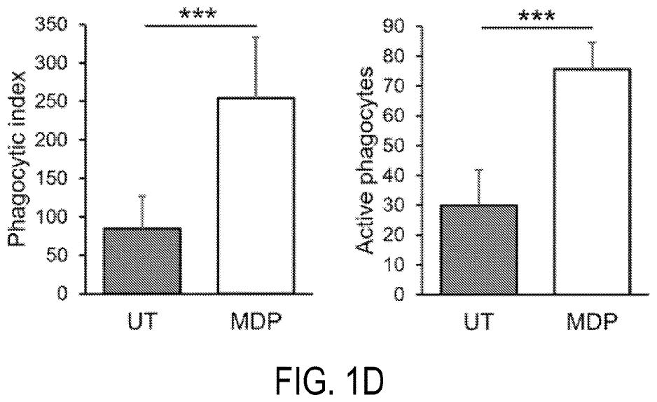

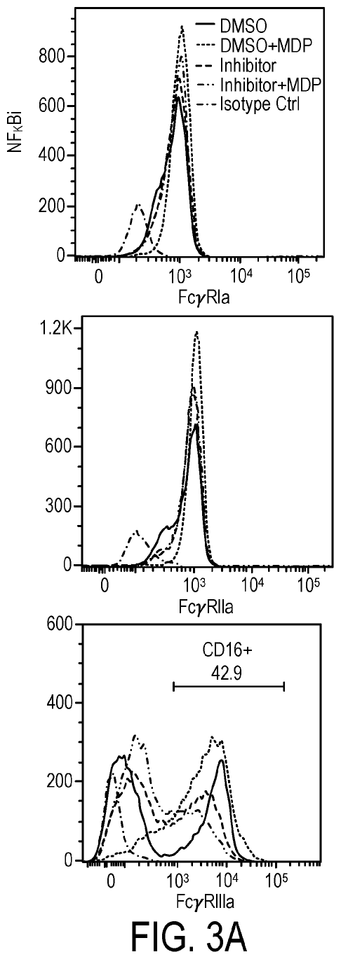

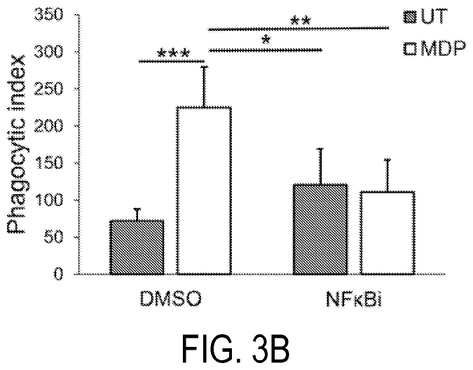

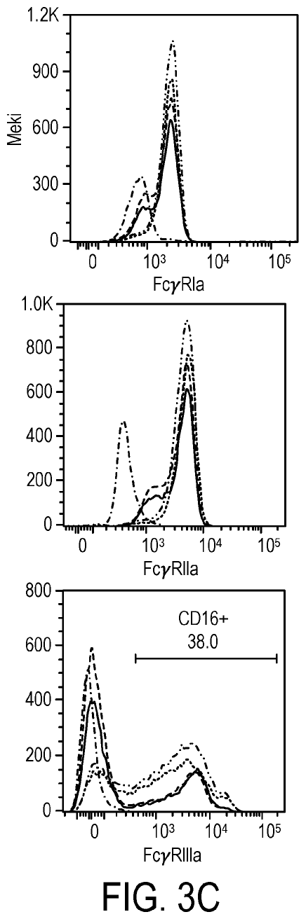

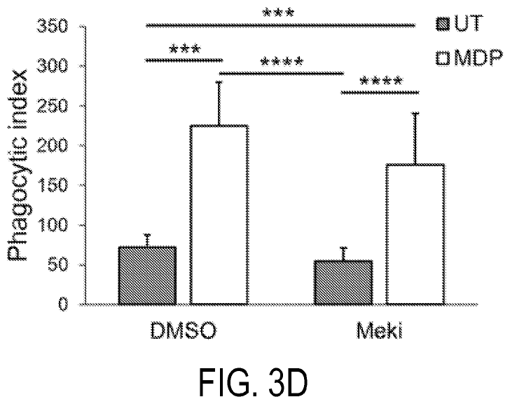

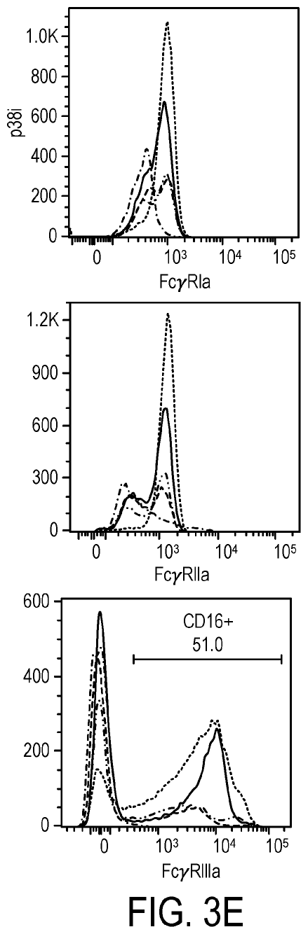

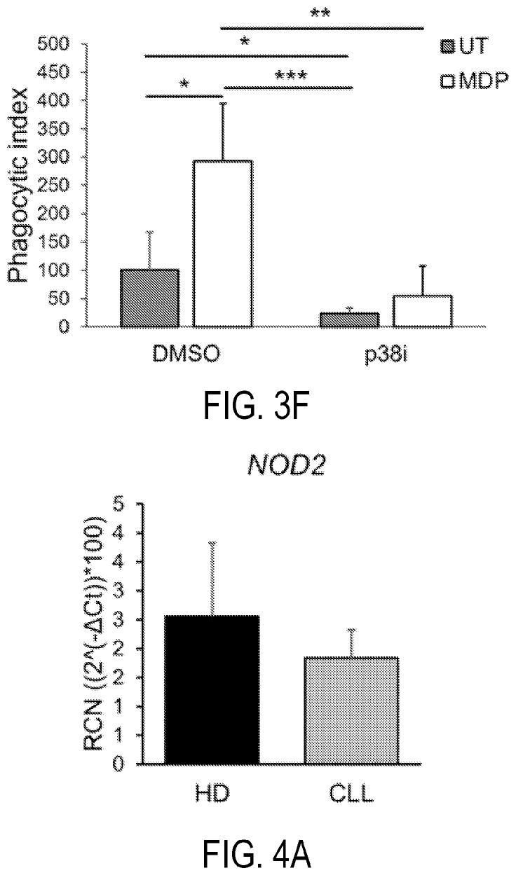

For measuring phagocytic activity, monocytes were treated with DMSO of pathway inhibitors for 30 minutes before stimulation with MDP at 1 μg/mL for 24 hours. Then, cells were washed and stained for contrast with carboxyfluorescein succinimidyl ester (CFSE, ThermoFisher Scientific) at 0.5 μM in PBS for 10 minutes at room temperature; the reaction was stopped with FBS and cells spun down. Cells were resuspended in complete media to perform the assay. Monocytes were mixed with 1 μL of packed fluorescently-labeled, opsonized sRBCs for 45 minutes at 37° C., then washed and fixed with paraformaldehyde at a final concentration of 1%. The amount of phagocytosed sRBCs was counted per 100 cells under the microscope. The counts were done independently by two or three different subjects in a blinded fashion and averages calculated. As control, phagocytosis of non-opsonized, fluorescent sRBCs was checked for negativity. FIGS. 1 and 3 were done using the same donors.

Mouse model. Mice were housed in a vivarium at The Ohio State University, following all institutional guidelines. All experimental procedures were approved in advance by the institution. An adoptive transfer Eμ-TCL1 mouse model of CLL was used. Briefly, 1×10⁷ splenocytes from CLL-burdened Eμ-TCL1 mice were used to engraft C57BL/6 mice. After 2 weeks of disease development, treatments (5 mg/kg of MDP and/or 1 mg/kg αCD20—Invivogen (San Diego, CA, USA, catalog #mcd20-mab10-) were delivered intraperitoneally three times weekly, for 2 weeks. Engrafted C57BL/6 mice, left untreated, and nongrafted C57BL/6 mice served as controls. Mice were euthanized using a CO₂ chamber, followed by cervical dislocation. Spleens and peripheral blood were collected. For spleens, the tissue was weighed and disrupted using a 100 m cell strainer (Corning); erythrocytes from both blood and disaggregated spleens were eliminated by centrifugation using lymphocyte centrifugation media. Cells were washed, resuspended in PBS and used for flow cytometry staining.

For flow cytometry staining, cells were placed in a v-bottom plate and stained as above using the antibodies listed for Panels 5 and 6 (Table 2). For intracellular staining we used the Foxp3 Intracellular staining kit (Thermo Fisher Scientific) according to the manufacturer's instructions as described previously, for Panel 6 (iNOS and EGR2). For acquisition, Absolute Count Beads (Biolegend) were used.

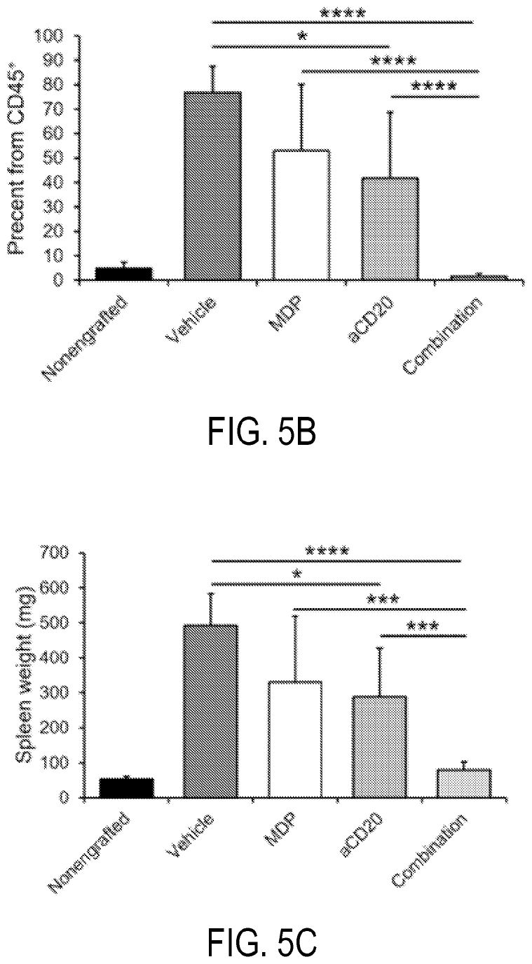

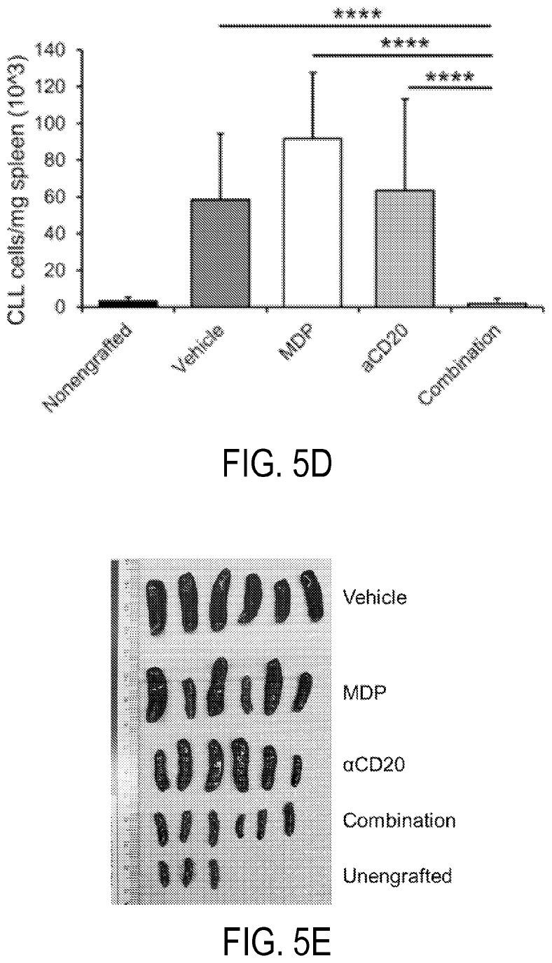

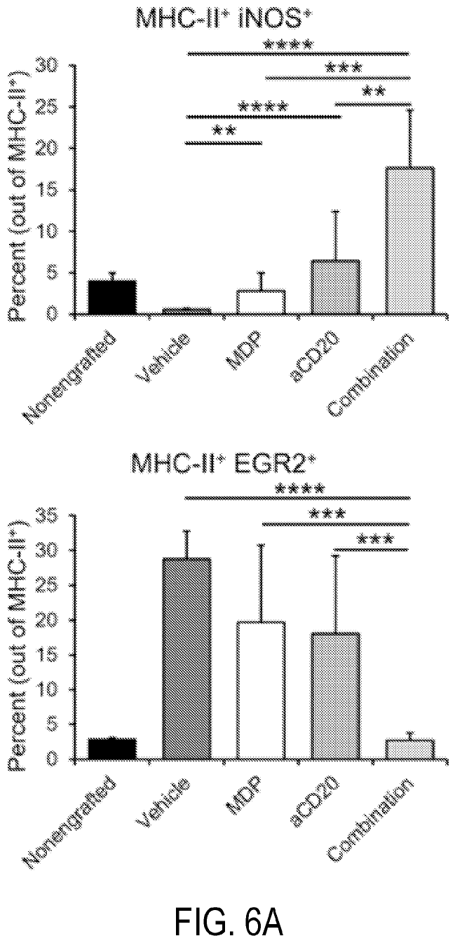

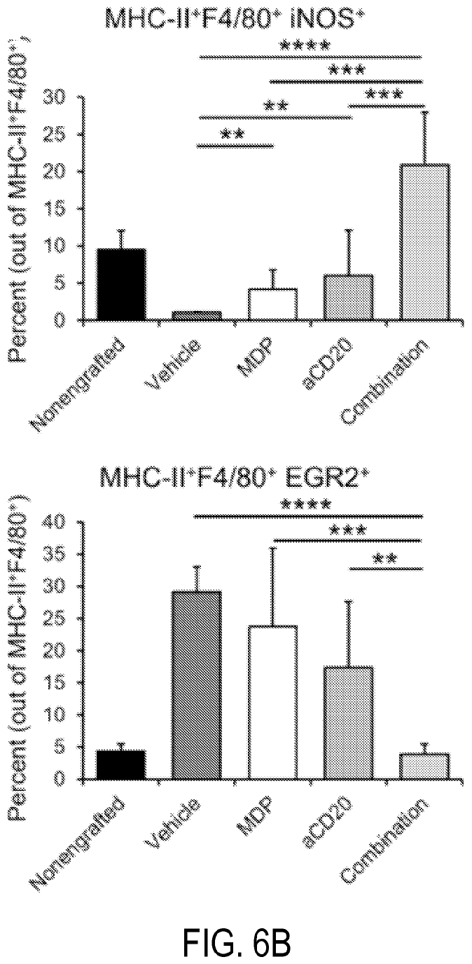

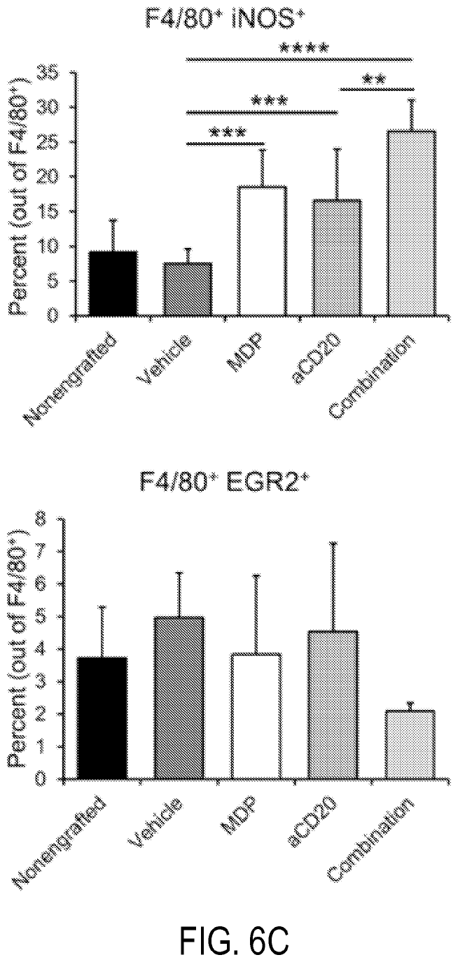

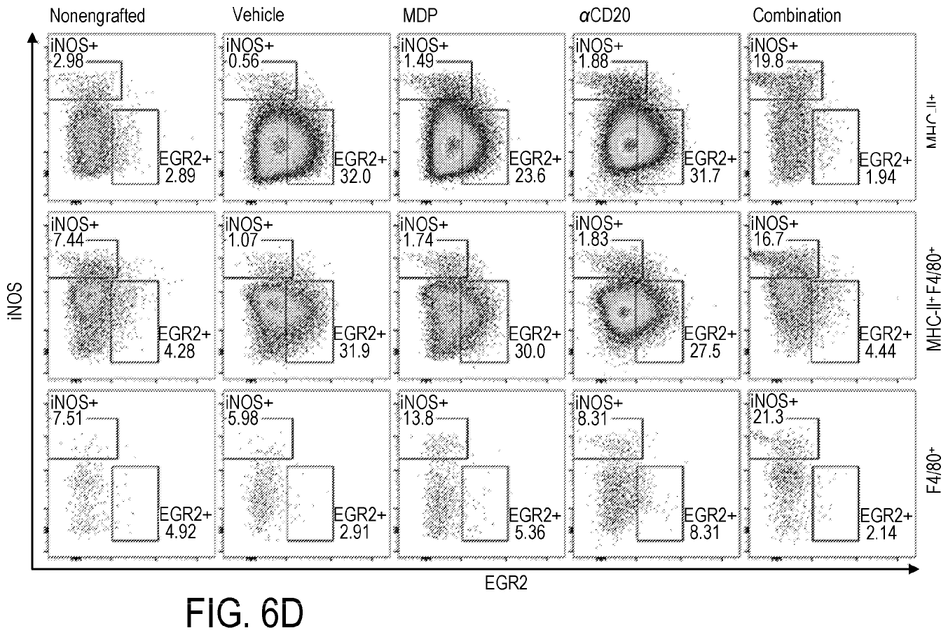

Statistical analysis. Graphs were prepared using Excel (Microsoft Corporation, Redmond, WA). For two-group comparisons, a one-tailed t-test analysis for paired samples was performed using Excel. For FIGS. 6, 9, and 10, data were analyzed with the mixed effects model using SAS 9.4 (SAS Institute, Cary, NC, USA). Statistical analyses included only the engrafted groups (vehicle, MDP, αCD20 and combination) for FIGS. 5 and 6; unengrafted controls were not compared.

### Results

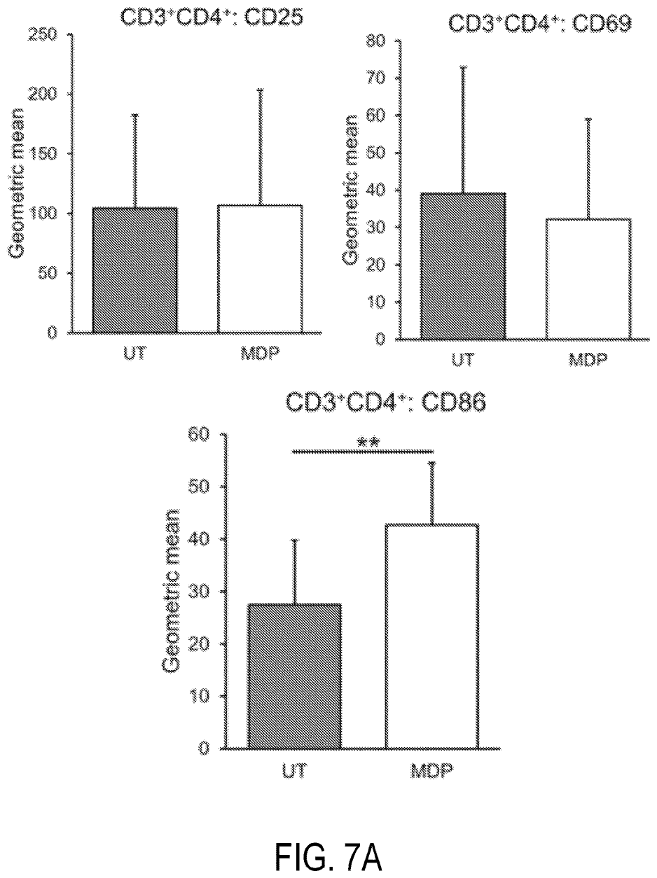

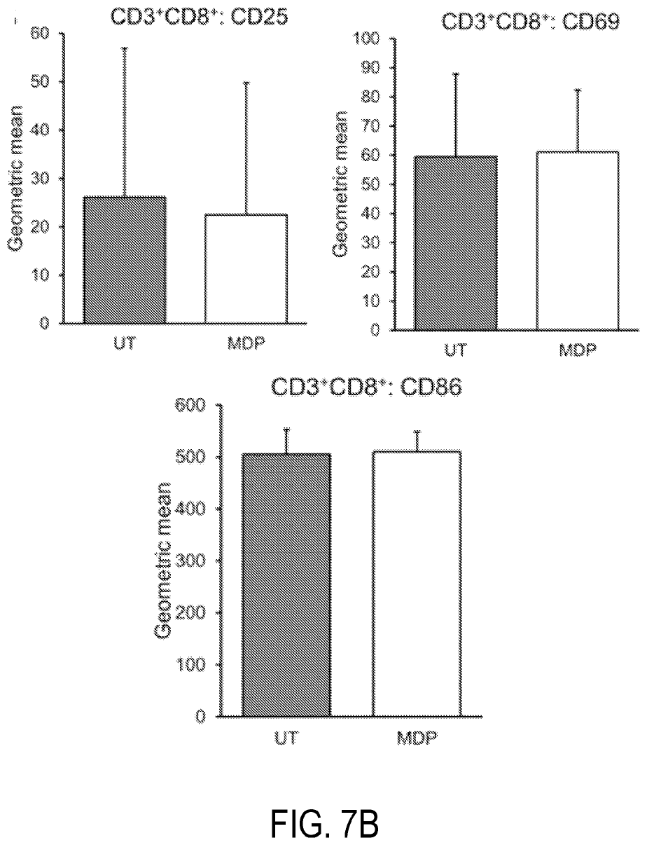

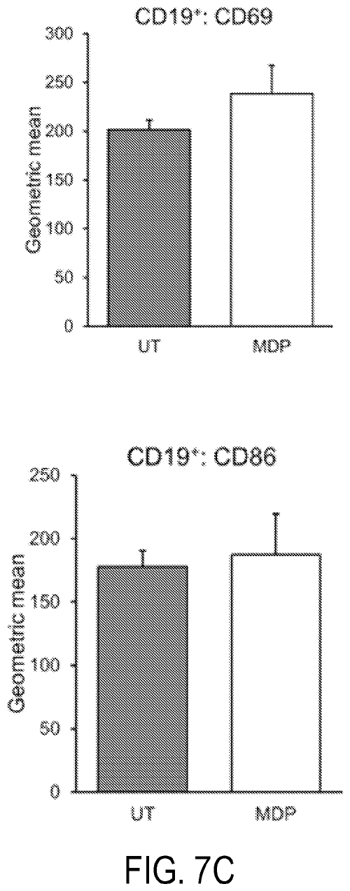

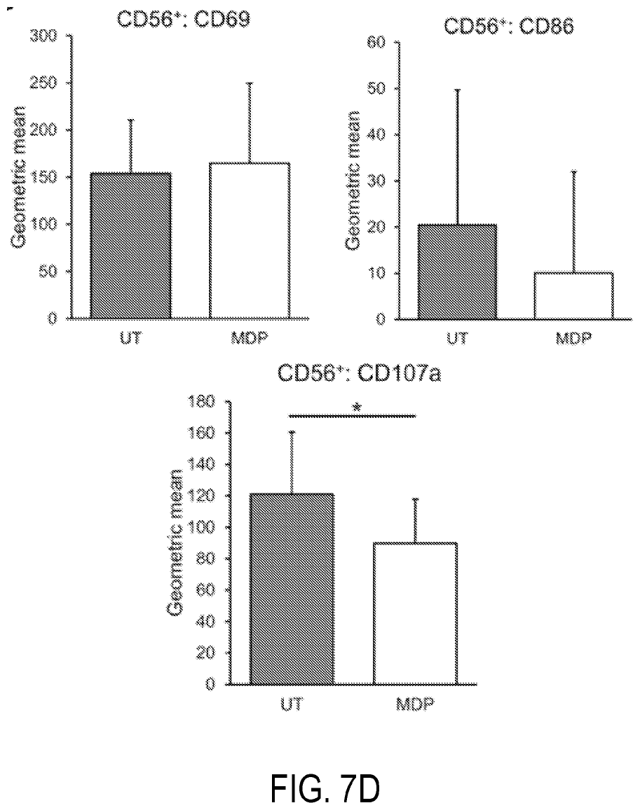

NOD2 agonists enhance monocyte FcγR function. Monocytes and macrophages are well-known responders to NOD2 agonists. However, the responsiveness of such cells from CLL patients is unknown. To test this, PBMCs from healthy donors were first treated with MDP at 1 mg/mL for 24 hours. Flow cytometry was then used to measure phenotypic markers. As shown in FIG. 1A, monocytes showed a significant increase in CD86 and HLA-DR. Concurrently there was a significant decrease in the M2-associated marker CD163. These results suggest that monocyte activation was skewed toward a proinflammatory M1-like phenotype. Markers were also measured in T cells, finding that NOD2 agonist treatment increased CD86 in CD4⁺ T cells (FIGS. 7A and 7B). B and NK cells were also examined but only a reduction in NK-cell CD107a was observed (FIGS. 7C and 7D). Upon further testing and in concordance with the change in markers (FIG. 1A), monocytes produced robust levels of TNFα in response to NOD2 stimulation (FIG. 1B). These results confirm earlier reports that monocytes are a major cell type responding to NOD2 agonists.

Next, it was tested to see whether NOD2 stimulation would increase the phagocytic ability of monocytes. For this monocytes were treated with MDP for 24 hours and then incubated them with fluoresceinated, antibody-opsonized sheep red blood cells (sRBCs). Here, cells were subjected to rosetting (initial binding stage of phagocytosis) and ingesting assays. Rosetting was done by incubating monocytes with the sRBCs at 4° C. while incubation at 37° C. was done in parallel to measure ingestion. As shown in FIG. 1C, there was a significant increase in binding (rosetting) in agonist-treated monocytes compared to controls. Likewise, the number of ingested sRBCs was significantly higher in treated monocytes (FIG. 1D). There was also a significant increase in the number of actively phagocytosing monocytes. Taken together these results indicate the NOD2 stimulation leads to an increase in FcγR-mediated phagocytosis.

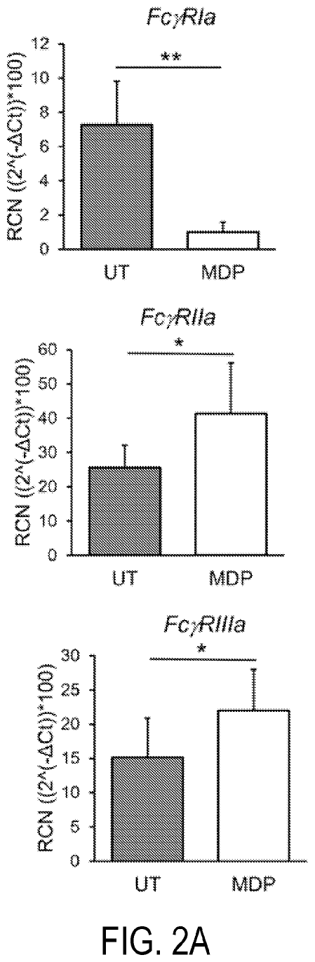

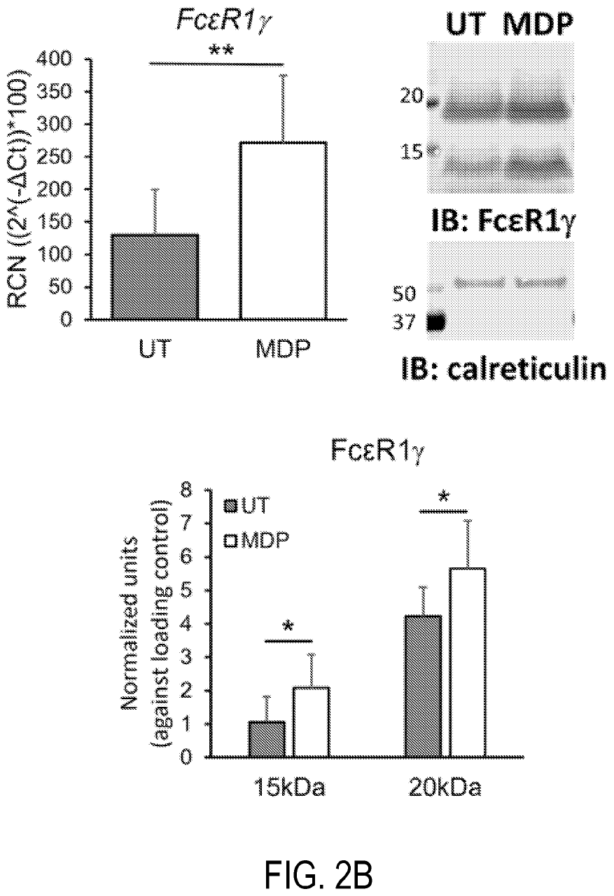

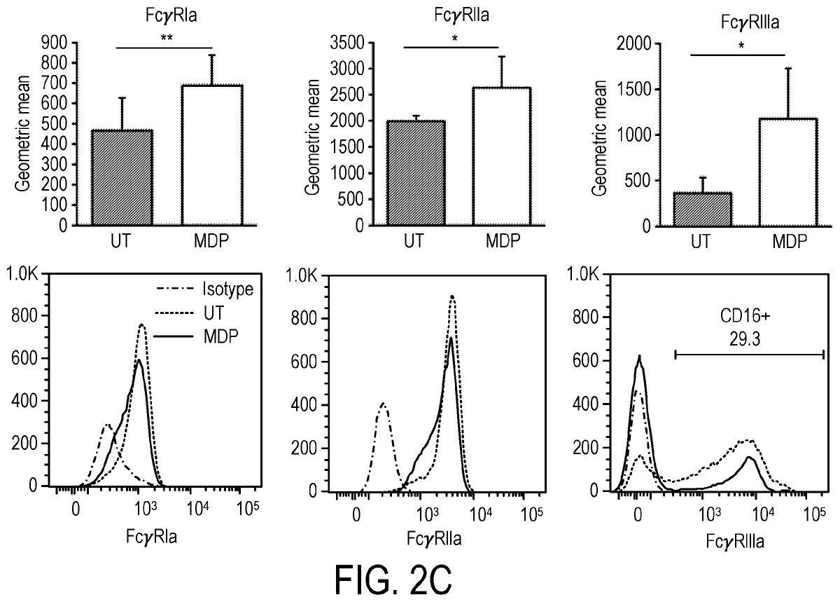

NOD2 stimulation modulates FcγR expression. To test whether the increase in phagocytosis could be accounted for by a change in FcγR expression, healthy-donor monocytes were treated with vehicle or MDP for 24 hours. Following this, transcripts for FcγRI, FcγRIIa, FcγRIIIa and FcγRIIb (the inhibitory FcγR) were evaluated by qRT-PCR. Results (FIG. 2A) showed that there was a significant increase in the expression of FcγRIIa and FcγRIIIa. The expression of FcγRI was significantly lower in treated monocytes. Changes in FcγRIIb expression were not significant (data not shown). Transcripts of FcεRIT were also significantly increased by MDP treatment (FIG. 2B, upper panel).

Surface expression of FcγR was also measured by flow cytometry. Consistent with an increase in transcripts, there was a significant increase in surface expression of FcγRIIa and FcγRIIIa (FIG. 2B). Surprisingly, the surface expression of FcγRI was also significantly increased despite the observed reduction in transcript (FIG. 2C). Because surface expression of FcγRI depends on the intracellular gamma-chain (FcεRIγ), protein levels of FcεRIγ were also measured using immunoblot analysis. As shown in FIG. 2B (middle panel and lower-panel graph), MDP significantly increased protein levels of FcεRIγ. Hence, despite reduced transcript of FcγRI, all three activating FcγR were expressed at greater levels on the cell surface following MDP treatment.

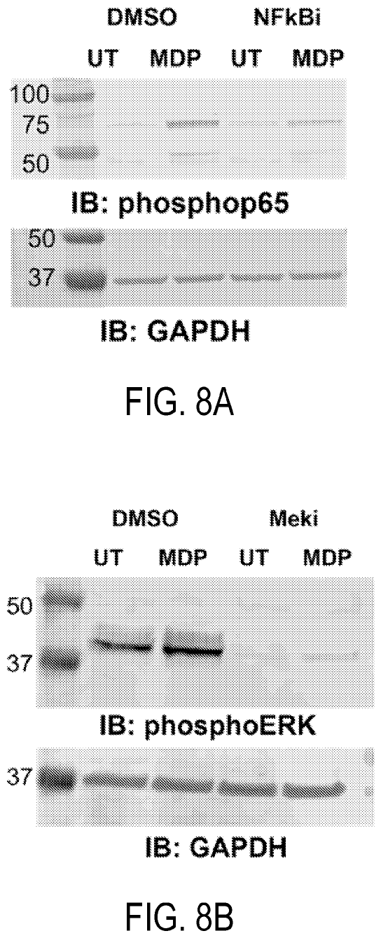

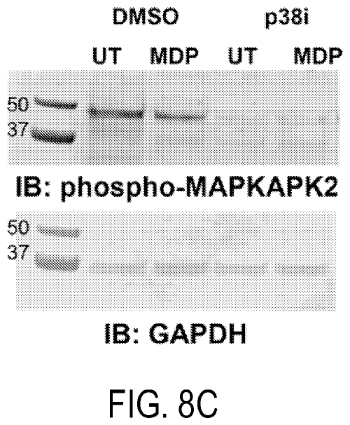

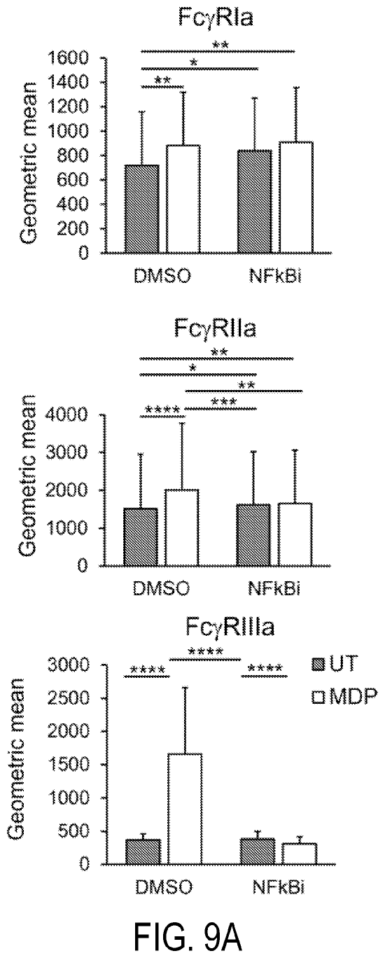

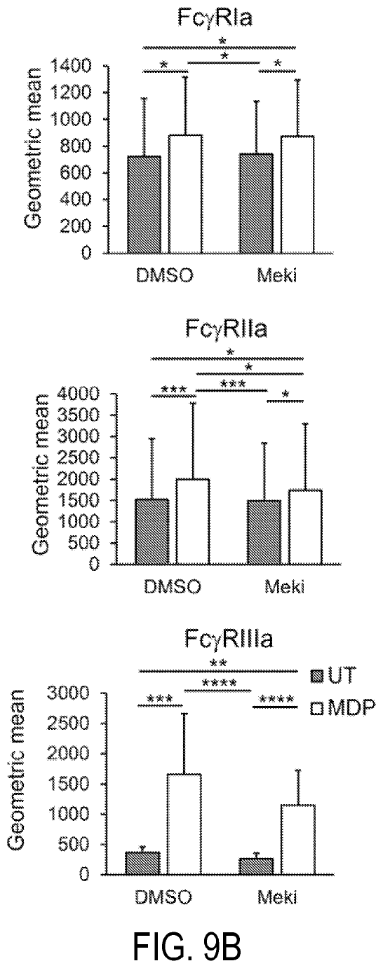

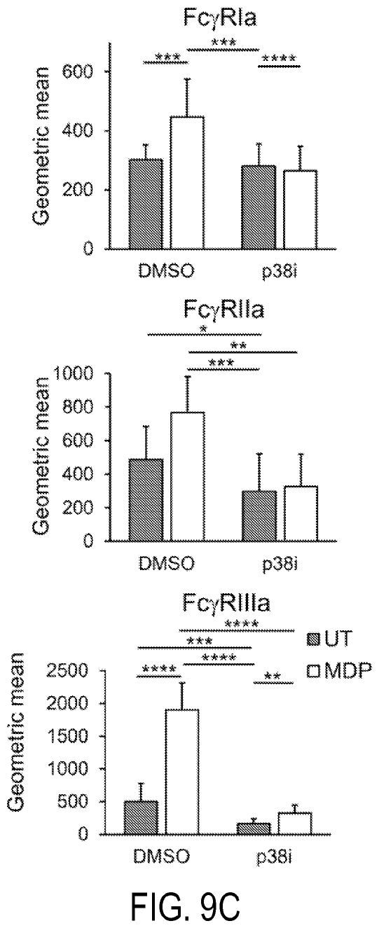

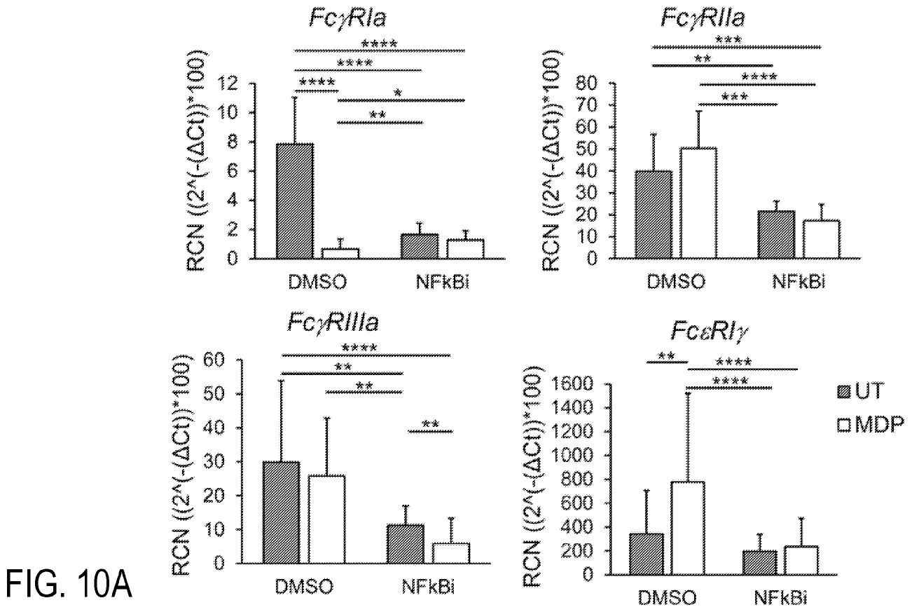

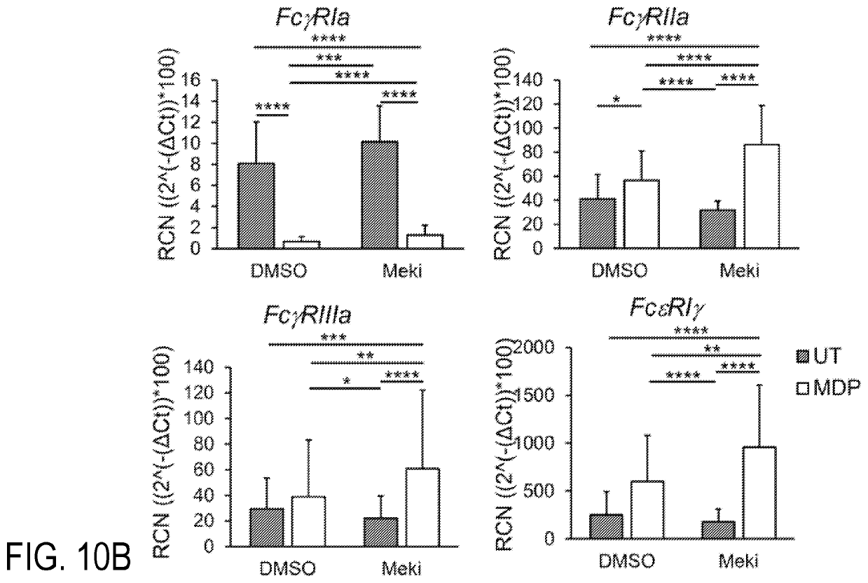

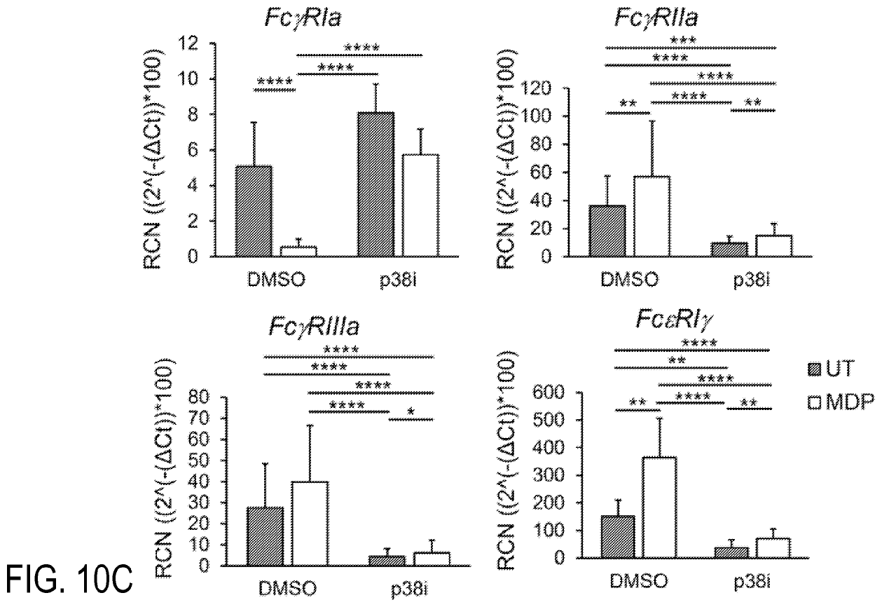

NF-κB and p38 are required for NOD2-mediated effects on FcγR. Previous studies have demonstrated that NF-κB, MEK and p38 are activated downstream of NOD2. Therefore, these pathways were explored to determined which ones were required for MDP-mediated changes in FcγR expression and function. For this, the three pathways were inhibited with pharmacologic inhibitors followed by treatment with vehicle or MDP. To verify the efficacy of the inhibitors used, immunoblot analysis was performed with phospho-specific antibodies for NF-κB, ERK and MAPKAPK2 (downstream of p38) (FIG. 8). qRT-PCR and flow cytometry were done to measure FcγR expression. As shown in FIG. 3A and quantified in FIG. 9, the increases in FcγR surface expression with MDP were prevented by inhibition of NF-κB and p38 but not MEK. Changes in transcript were also largely prevented by these two inhibitors (FIG. 10). Consistent with the effects of these inhibitors on changes in FcγR expression, the MDP-mediated increase in phagocytosis was prevented with NF-κB and p38 inhibition (FIG. 3B). Taken together, these data demonstrate that NF-κB and p38 are the required mediators for enhancing FcγR expression and function downstream of NOD2.

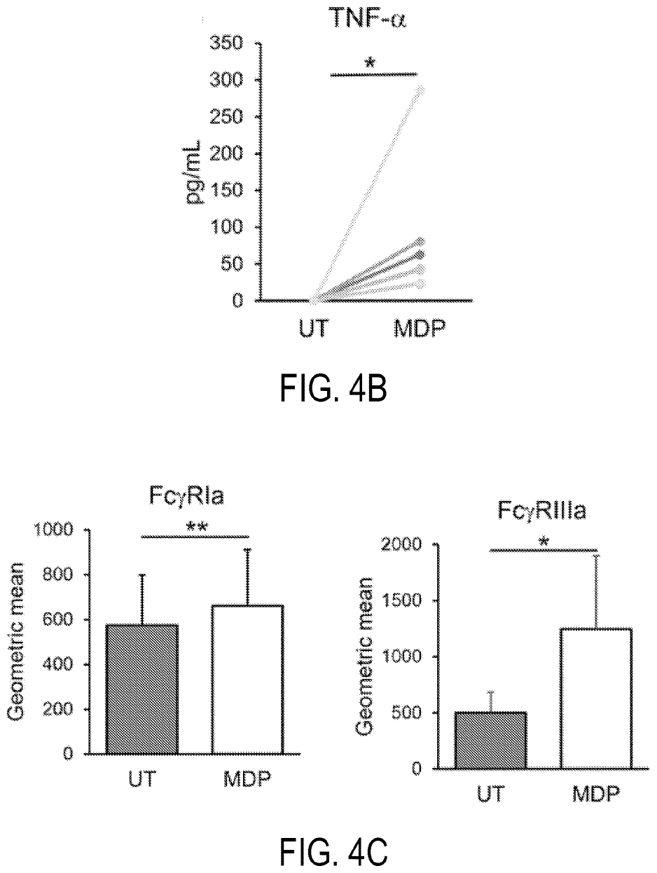

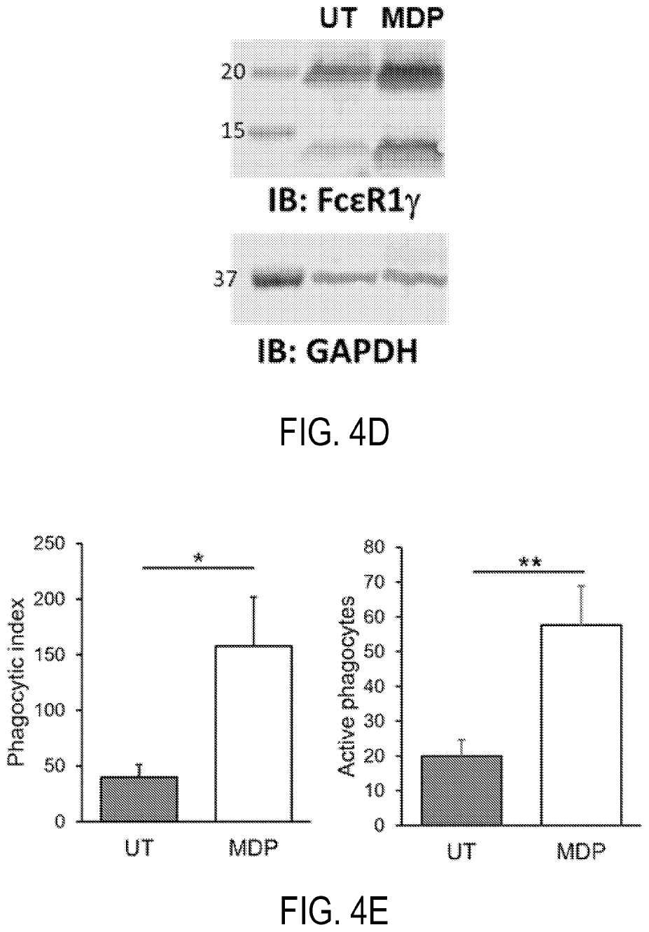

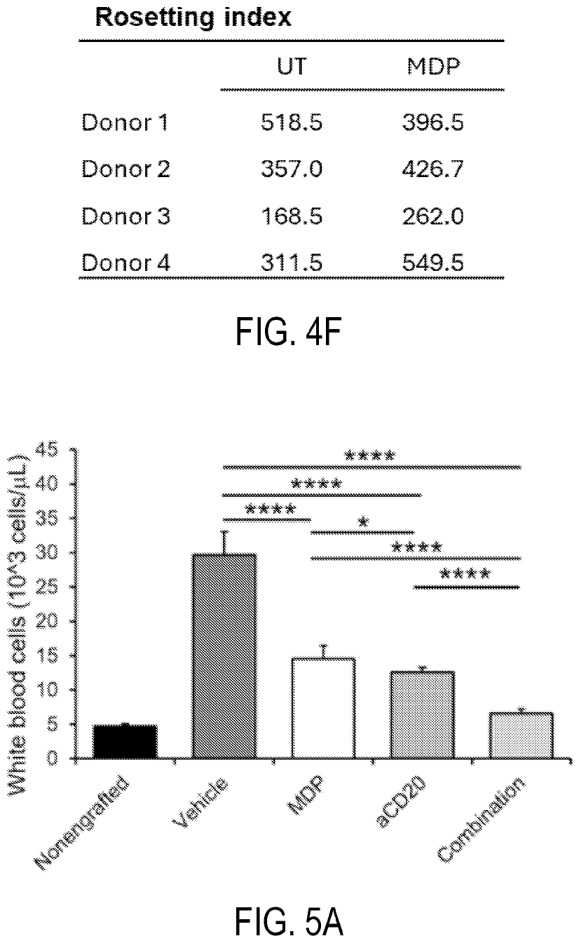

NOD2 regulates FcγR function in CLL-patients monocytes. Next, to test whether the effects of NOD2 seen in healthy-donor monocytes were reproducible in CLL-patient monocytes, NOD2 expression and function was first measured. As shown in FIGS. 4A and 4B, NOD2 is expressed in CLL monocytes at slightly but not significantly lower levels than in healthy-donor monocytes. Patient-derived monocytes were able to respond to MDP by producing TNFα. MDP treatment of CLL monocytes also enhanced surface expression of FcγRI, FcγRIIIA (FIG. 4C) and expression of FcεR1γ(FIG. 4D). In contrast to healthy-donor monocytes, there was no change in FcγRIIa in patient-derived monocytes after MDP treatment (data not shown). However, consistent with an overall increase in activating FcγRs, MDP treatment led to a significantly higher level of phagocytosis (FIG. 4E). In contrast, the same increase in rosetting was not seen in patient monocytes (FIGS. 4E and 4F). These results show that CLL-patient monocyte responses to MDP are measurable but muted compared to those seen with healthy-donor cells.

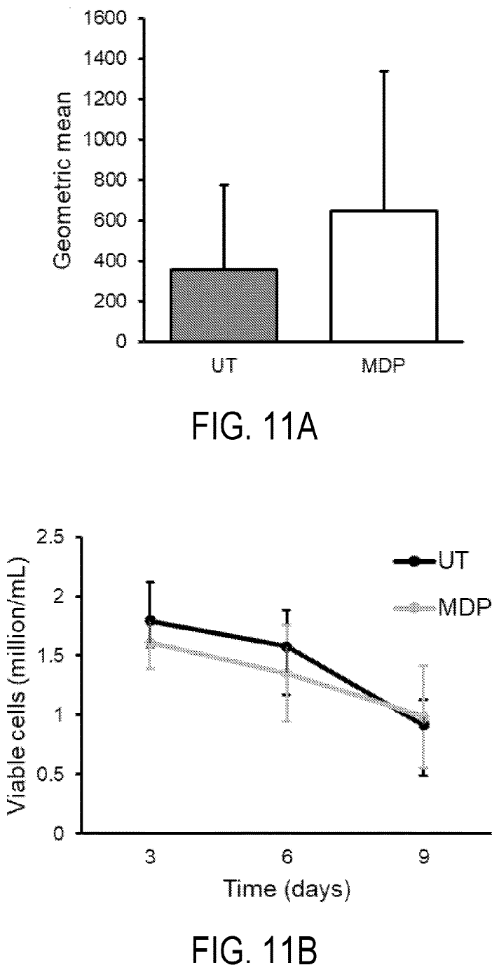

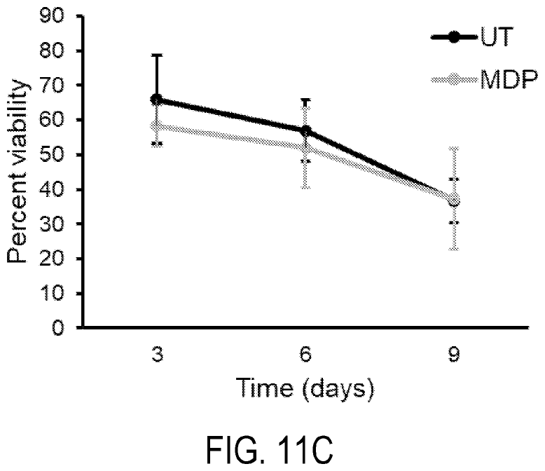

In addition to monocytes, the effect of MDP on B/CLL cells in patient samples was examined. PBMCs from CLL patients were treated with MDP for 24 hours, then expression of CD86 in B/CLL cells was measured using flow cytometry. Although there was some increase in CD86, it was not significant (FIG. 11A). B/CLL cells were isolated and treated with vehicle or MDP, counting cell numbers and viability at different time points. As shown in FIGS. 11B and 11C, the viability of CLL cells was not affected by MDP. These results show that NOD2 activation does not affect cell viability. Taking cell number and percent viability together, results also show that MDP did not affect CLL-cell proliferation.

NOD2 activation enhances effects of antitumor antibody in vivo. To test the effects of NOD2 stimulation on FcγR function in vivo, a mouse model that mimics human CLL was used. C57/BL6 mice were engrafted with splenocytes from diseased Eμ-TCL1 mice, as described in materials and methods. Disease was allowed to develop for 2 weeks. Animals were either left untreated or treated with MDP alone, αCD20 antibody alone or a combination of the two. Mice were sacrificed after 2 weeks of treatment. Healthy, unengrafted mice were included as controls. CLL loads in blood and spleen were assessed. White blood cell (WBC) counts in blood were significantly reduced by each single treatment, and dual treatment showed greater reduction than either single treatment (FIG. 5A). Similarly, there was a significantly lower percentage of CLL cells after dual treatment compared to either single treatment (FIG. 5B).

Spleen weights were significantly lower in MDP plus antibody mice (FIG. 5C) and the amount of CLL cells per mg of spleen (FIG. 5D) was also significantly lower with dual treatment. An image of the spleens is shown in FIG. 5E. These results show that the combination of NOD2 agonist plus therapeutic antibody may be more effective than antibody alone.

NOD2 agonists enhance antibody-mediated monocyte macrophage activation in vivo. In addition to the leukemic burden, it was tested whether MDP combined with antitumor antibody could modulate the phenotypes of monocytes and macrophages in vivo. For this, cells from the spleen were obtained and stained for flow cytometry to detect M1- and M2-related markers on monocytes and macrophages. Cells were gated as CD45⁺CD11b⁺Ly6G⁻ and further classified by expression of MHC-II and/or F4/80. Percentages of cells expressing iNOS (M1) or EGR2 (M2)(PMID: 26699615) were measured to assess M1 or M2 polarization. Results from unengrafted, healthy control mice were also collected.

In MHC-II⁺ populations either negative or positive for F4/80 (mainly monocytes or macrophages, FIGS. 6A and 6B) there was a strong and significant upregulation of iNOS and downregulation of EGR2, showing a shift toward an M1-like phenotype. Increased iNOS was seen in the F4/80⁺ group (FIG. 6C). Taken together, these data show that MDP/antibody combination treatment not only decreases CLL burden but also shifts myeloid phenotypes toward a more proinflammatory, M1-like state.

### DISCUSSION

The present disclosure shows for the first time NOD2 agonists used to offset the suppressive leukemic microenvironment and enhance antibody-mediated responses in CLL. MDP led to enhanced responses in vitro, although these responses were measurably stronger in healthy-donor cells than cells from CLL patients. In vivo, MDP significantly enhanced the activity of αCD20 antibody, leading to lower tumor burden and a shift toward an M1-like phenotype.

The activation of monocytes in both healthy-donor and CLL-patient monocytes after NOD2 stimulation led to a significant increase in the expression of activating FcγRs. Previously, it has been shown that NOD2 promotes a phenotypic change from classical to non-classical monocytes, which involves the increase of FcγRIIIa. This observation is in line with the present disclosure and, in addition, a significant increase in the surface expression of FcγRia was observed, likely being stabilized by the common gamma chain. The increase of expression of activating FcγRs in the membrane is of importance since patients with active CLL have a dominance of FcγRIIb signaling. In addition, FcγRs are crucial for αCD20 antibody efficacy in vivo. Thus, using a NOD2 agonist in combination with a therapeutic antibody may overcome resistance to this therapeutic strategy. This is supported further by the increase in phagocytosis seen in both healthy-donor and CLL-patient monocytes, which is one of the main mechanisms for elimination of αCD20-opsonized targets by myeloid cells.

The combination of NOD2 stimulation with a therapeutic antibody against CD20 was observed to not only influence the antibody-mediated responses, but also myeloid cells within the microenvironment. Using the adoptive transfer EμTCL-1 mouse model, a significant decrease of the disease burden was seen in mice treated with the combination, in comparison with either single treatment alone. Most importantly, an increase of monocytes or macrophages that expressed iNOS was also observed. This was seen when mice were treated with both MDP and αCD20 antibody, but not with either treatment alone, showing that dual treatment was needed to overcome the suppressive environment. NOS2 or iNOS has been widely attributed to M1-related IFNγ stimulation, while higher EGR2 expression is highly correlated with M2-like responses. Thus, the iNOS response to dual treatment shows an active switch in the phenotype of myeloid cells, which may involve the activation of NF-κB or MAPK, or an increase in IFNγ production.

It has been previously shown that NOD2 activation may result in a variety of outcomes in myeloid cells, depending on the diseases studied and the agonists used. Mifamurtide has been shown to induce monocyte activation in treated patients, which involved production of cytokines and direct cytotoxicity against tumoral cell lines. In addition, macrophages polarized in vitro with GM-CSF and further activated with MTP-PE and IFNγ are capable of inhibiting osteosarcoma growth by producing soluble factors, however independent of TNFα or IL 1-β. Dieter et al. showed that MTP-PE could induce TNFα and nitric oxide from liver macrophages and that MTP-PE-stimulated macrophages showed direct cytotoxic effects against the P815 mouse mastocytoma cell line (Dieter P, Hempel U, Kamionka S, Kolada A, Malessa B, Fitzke E, et al. Prostaglandin E2 affects differently the release of inflammatory mediators from resident macrophages by LPS and muramyl tripeptides. Mediators of inflammation. 1999; 8(6):295-303). The present disclosure demonstrates of TNFα production following MDP treatment, which supports these observations.

There is concern that MDP may have effects apart from promoting myeloid-cell activation. A previous report showed that MDP increased activation and survival of CLL cells in a specific group of patients, which may be an undesired side effect. However, a substantial activation of B cells in healthy donors or CLL patients was not seen herein, either as isolated cells or in PBMCs cultures. Although results may differ due to the use of a different stimulation approaches, this observation shows that the use of NOD2 agonists for CLL treatment may not result in leukemic progression.

Along with this, the benefits of NOD2 agonists seem to outweigh such unwanted effects when used against other types of cancer. Mifamurtide (muramyl tripeptide phosphatidylethanolamine, MTP-PE in its encapsulated form), has been used for the treatment of osteosarcoma in Europe. In 2009, the European Medicines Agency granted Takeda marketing approval, based on the INT-0133 clinical trial. The trial examined the effects of MTP-PE with chemotherapeutic agents, finding that MTP plus a four-drug chemotherapy regimen (doxorubicin, ifosfamide, cisplatin and methotrexate) showed the best event-free survival. Further studies have been done to complement observations from the INT-0133 trial. The use of this NOD2 agonist is approved for youth populations (2 to 30 years of age) as follow-up treatment after surgical removal of the main affected tissue.

In conclusion, the present disclosure provides evidence that combination therapy of αCD20 antibody and NOD2 agonists is useful to overcome antibody resistance and to promote the elimination of leukemic cells in CLL. This is also of benefit for patients who acquired resistance to BTK inhibitors or to other therapies.

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

### Tables

TABLE 1
Real-time PCR primers used.

| | Forward | Reverse |
|---|---|---|
| GAPDH | ACTTTGGTATCGTGGAAGGACT | GTAGAGGCAGGGATGATGTTCT |
| | (SEQ ID NO: 1) | (SEQ ID NO: 2) |
| FcγRIa | GGCAAGTGGACACCACAAAGGCA | GCTGGGGGTCGAGGTCTGAGT |
| | (SEQ ID NO: 3) | (SEQ ID NO: 4) |
| FcγRIIa | TTGCTGCTGCTGGCTTCTGC | GTAGCTGGGCTGCGTGTGGG |
| | (SEQ ID NO: 5) | (SEQ ID NO: 6) |
| FcγRIIIa | CCTCTCCACCCTCAGTGACCCG | TGGAGCAACAGCCAGCCGAT |
| | (SEQ ID NO: 7) | (SEQ ID NO: 8) |
| FcεR1γ | CAAGCAGCGGCCCTGGGAG | TTCCTGGTGCTCAGGCCCGT |
| | (SEQ ID NO: 9) | (SEQ ID NO: 10) |

TABLE 2
Flow cytometry panels

| Panel | Cocktail |
|---|---|
| Panel 1: Monocyte activation | CD14 Alexa Flour 647 (clone HCD14, Biolegend) |
| | CD38 FITC (clone HIT2, Biolegend |
| | CD163 PeCy7 (clone GHI/61, Biolegend) |
| | CD206 Brilliant Violet 605 (clone 15-2, Biolegend) |
| | CD86 PE (clone BC96, Biolegend) |
| | HLA-DR PercP (clone L243, Biolegend) |
| | Live/Dead Blue (ThermoFisher Scientific) |
| Panel 2: T cell activation | CD3 Pacific Blue (clone OKT3, Biolegend) |
| | CD4 Brilliant Violet 711 (clone RPA-T4, Biolegend) |
| | CD8 PercP (clone SK1, Biolegend) |
| | CD69 Alexa Fluor 488 (clone FN50, Biolegend) |
| | CD25 PeCy7 (clone BC96, Biolegend) |
| | CD86 APC (clone IT2.2, Biolegend) |
| | Live/Dead Blue |
| Panel 3: B and NK cell | CD19 PeCy7 (clone HIB19, Invitrogen) |
| activation | CD69 Alexa Flour 488 |
| | CD86 APC |
| | CD56 Super Bright 600 (clone NCAM, Invitrogen) |
| | CD107a PE/Dazzle (clone H4A3, Invitrogen) |
| | Live/Dead Blue |
| Panel 4: FcγRs | CD64 (FcγRIa) PercP Cy5.5 (clone 10.1, Biolegend) |
| | CD32a (FcγRIIa) FITC (clone IV.3, StemCell Technologies) |
| | CD32b (FcγRIIb) PeCy7 (clone S18005H, Biolegend) |
| | CD16 (FcγRIIIa) Brilliant Violet 711 (clone 3G8, |
| | Biolegend) |
| | Live/Dead Blue |
| Panel 5: CLL cells (Mouse | CD45 Brilliant Violet 421 (clone 30-F11, Biolegend) |
| model) | CD19 PE (clone 1D3/CD19, Biolegend) |
| | CD5 Alexa Fluor 647 (clone 53-7.3, Biolegend) |
| | Live/Dead Blue. |
| Panel 6: Monocyte/macrophage | CD45 Brilliant Violet 421 |
| phenotype (Mouse model) | CD11b Brilliant Violet 711 (clone M1/70, Biolegend) |
| | Ly6G PerCP (clone 1A8, Biolegend) |
| | Ly6C PeCy7 (clone HK1.4, Biolegend) |
| | F4/80 Brilliant Violet 605 (clone BM8, Biolegend) |
| | IA-IE Brilliant Violet 785 (clone M5/114.15.2, Biolegend) |
| | iNOS PE (clone CXNFT, Invitrogen) |
| | EGR2 APC (clone Erongr2, Invitrogen) |
| | Live/Dead Blue |

### Sequences

1.
-GAPDH Forward Primer
SEQ ID NO: 1
ACTTTGGTATCGTGGAAGGACT
2.
-GAPDH Reverse Primer
SEQ ID NO: 2
GTAGAGGCAGGGATGATGTTCT
3.
-FcγRIa Forward Primer
SEQ ID NO: 3
GGCAAGTGGACACCACAAAGGCA
4.
-FcγRIa Reverse Primer
SEQ ID NO: 4
GCTGGGGGTCGAGGTCTGAGT
5.
-FcγRIIa Forward Primer
SEQ ID NO: 5
TTGCTGCTGCTGGCTTCTGC
6.
-FcγRIIa Reverse Primer
SEQ ID NO: 6
GTAGCTGGGCTGCGTGTGGG
7.
-FcγRIIIa Forward Primer
SEQ ID NO: 7
CCTCTCCACCCTCAGTGACCCG
8.
-FcγRIIIa Reverse Primer
SEQ ID NO: 8
TGGAGCAACAGCCAGCCGAT
9.
-FcεR1γ Forward Primer
SEQ ID NO: 9
CAAGCAGCGGCCCTGGGAG
10.
-FcεR1γ Reverse Primer
SEQ ID NO: 10
TTCCTGGTGCTCAGGCCCGT

1. A method of treating chronic lymphocytic leukemia (CLL), the method comprising administering to a subject in need thereof a therapeutically effective amount of a nucleotide-binding oligomerization domain-containing protein 2 (NOD2) agonist and an immunotherapeutic agent, wherein the method decreases tumor burden of CLL relative to an untreated control.

2. The method of claim 1, wherein the NOD2 agonist comprises a muramyldipeptide (MDP), a muramyltripeptide (MTP), L18-MDP, M-TriLYS, murabutide, N-Glycolyl-MDP, or a derivative thereof.

3. The method of claim 1, wherein the immunotherapeutic agent comprises a monoclonal antibody.

4. The method of claim 3, wherein the monoclonal antibody binds to a tumor cell and interacts with a constant fragment (Fc) on an effector cell.

5. The method of claim 1, wherein the NOD2 agonist and the immunotherapeutic agent are administered concurrently.

6. The method of claim 1, wherein the NOD2 agonist and the immunotherapeutic agent are administered consecutively.

7. The method of claim 1, wherein the NOD2 agonist and the immunotherapeutic agent increases an antibody-mediated response relative to the immunotherapeutic agent alone.

8. The method of claim 7, wherein the antibody-mediated response comprises monocyte or macrophage activation.

9. The method of claim 1, wherein the method shifts a monocyte or a macrophage from an M2-like phenotype to an M1-like phenotype.

10. The method of claim 1, further comprising administering an adjuvant, an additive, or a combination thereof.

11. The method of claim 10, wherein the adjuvant, additive, or a combination are administered concurrently or consecutively with the NOD2 agonist and the immunotherapeutic agent.

12. A The method of claim 1, wherein the nucleotide-binding oligomerization domain-containing protein 2 (NOD2) agonist and immunotherapeutic agent are in a composition, and further wherein the composition further comprises a pharmaceutically acceptable carrier.

13-19. (canceled)

20. The method of claim 12, wherein the pharmaceutically acceptable carrier comprises an excipient, a diluent, a filler, a salt, a buffer, a stabilizer, a solubilizer, a lipid, a stabilizer, or any combination thereof.

21. A method of stimulating a monocyte or a macrophage, the method comprising exposing the monocyte or the macrophage to an effective amount of a nucleotide-binding oligomerization domain-containing protein 2 (NOD2) agonist and an immunotherapeutic agent, wherein the method shifts the monocyte or the macrophage from an M2-like phenotype to an M1-like phenotype.

22. The method of claim 21, wherein the NOD2 agonist comprises a muramyldipeptide (MDP), a muramyltripeptide (MTP), L18-MDP, M-TriLYS, murabutide, N-Glycolyl-MDP, or a derivative thereof.

23. The method of claim 21, wherein the immunotherapeutic agent comprises a monoclonal antibody, and further wherein the monoclonal antibody binds to a tumor cell and interacts with a constant fragment (Fc) on an effector cell.

24. (canceled)

25. The method of claim 21, further comprising exposing the monocyte or macrophage to an adjuvant, an additive, or a combination thereof.

26. An immunotherapeutic composition comprising:
a muramyldipeptide (MDP), a muramyltripeptide (MTP), L18-MDP, M-TriLYS, murabutide, N-Glycolyl-MDP, or a derivative thereof;
a monoclonal antibody; and
a pharmaceutically acceptable carrier.

27. The composition of claim 26, further comprising an adjuvant, an additive, or a combination thereof.

28. The composition of claim 26, wherein the pharmaceutically acceptable carrier comprises an excipient, a diluent, a filler, a salt, a buffer, a stabilizer, a solubilizer, a lipid, a stabilizer, or any combination thereof.


SEQUENCE LISTING (10 sequences)

SEQ ID NO: 1 - DNA, 22 bp, synthetic construct
  misc_feature 1..22: note = Description of sequence: GAPDH Forward Primer
  source 1..22: mol_type = other DNA
actttggtatcgtggaaggact

SEQ ID NO: 2 - DNA, 22 bp, synthetic construct
  misc_feature 1..22: note = Description of sequence: GAPDH Reverse Primer
  source 1..22: mol_type = other DNA
gtagaggcagggatgatgttct

SEQ ID NO: 3 - DNA, 23 bp, synthetic construct
  misc_feature 1..23: note = Description of sequence: Fc-gamma-RIa Forward Primer
  source 1..23: mol_type = other DNA
ggcaagtggacaccacaaaggca

SEQ ID NO: 4 - DNA, 21 bp, synthetic construct
  misc_feature 1..21: note = Description of sequence: Fc-gamma-RIa Reverse Primer
  source 1..21: mol_type = other DNA
gctgggggtcgaggtctgagt

SEQ ID NO: 5 - DNA, 20 bp, synthetic construct
  misc_feature 1..20: note = Description of sequence: Fc-gamma-RIIa Forward Primer
  source 1..20: mol_type = other DNA
ttgctgctgctggcttctgc

SEQ ID NO: 6 - DNA, 20 bp, synthetic construct
  misc_feature 1..20: note = Description of sequence: Fc-gamma-RIIa Reverse Primer
  source 1..20: mol_type = other DNA
gtagctgggctgcgtgtggg

SEQ ID NO: 7 - DNA, 22 bp, synthetic construct
  misc_feature 1..22: note = Description of sequence: Fc-gamma-RIIIa Forward Primer
  source 1..22: mol_type = other DNA
cctctccaccctcagtgacccg

SEQ ID NO: 8 - DNA, 20 bp, synthetic construct
  misc_feature 1..20: note = Description of sequence: Fc-gamma-RIIIa Reverse Primer
  source 1..20: mol_type = other DNA
tggagcaacagccagccgat

SEQ ID NO: 9 - DNA, 19 bp, synthetic construct
  misc_feature 1..19: note = Description of sequence: Fc-episilion-R1-gamma Forward Primer
  source 1..19: mol_type = other DNA
caagcagcggccctgggag

SEQ ID NO: 10 - DNA, 20 bp, synthetic construct
  misc_feature 1..20: note = Description of sequence: Fc-episilion-R1-gamma Reverse Primer
  source 1..20: mol_type = other DNA
ttcctggtgctcaggcccgt